(19) United States
(12) Patent Application Publication
(10) Pub. No.: US 2023/0316431 A1
(43) Pub. Date: Oct. 5, 2023

(54) INFORMATION PROCESSING DEVICE, INFORMATION PROCESSING METHOD, AND PROGRAM

(71) Applicant: Sony Group Corporation, Tokyo (JP)

(72) Inventor: Masatoshi Funabashi, Tokyo (JP)

(73) Assignee: Sony Group Corporation, Tokyo (JP)

(21) Appl. No.: 18/016,271

(22) PCT Filed: Jul. 7, 2021
(86) PCT No.: PCT/JP2021/025617

§ 371 Date: Jan. 13, 2023

(30) Foreign Application Priority Data
Jul. 20, 2020 (JP) 2020-123762

(51) Int. Cl. G06Q 50/02; A01B 79/00; G16H 20/90

(52) U.S. Cl. CPC G06Q 50/02; A01B 79/005; G16H 20/90

(57) ABSTRACT

The present technology relates to an information processing device, an information processing method, and a program that are capable of providing an appropriate cultivation condition for cultivating a crude drug by using a diversity-promoting cultivation method. A cultivation condition for cultivating a specific crude drug is identified with the use of a first model that associates a crude drug with a cultivation condition for cultivating the crude drug by a diversity-promoting cultivation method for promoting biodiversity and controlling an ecosystem to produce vegetation. For example, the present technology is applicable to a case where a crude drug to be used to produce an herbal medicine is cultivated by using synecoculture (registered trademark) or the like.

### TECHNICAL FIELD

The present technology relates to an information processing device, an information processing method, and a program, and more particularly to an information processing device, an information processing method, and a program that are capable of providing an appropriate cultivation condition for cultivating vegetation such as a crude drug by using a diversity-promoting cultivation method which promotes biodiversity and controls an ecosystem to produce plants.

### BACKGROUND ART

In recent years, synecoculture (registered trademark) has been attracting attention as a farming method based on species diversity which exceeds a natural state as a result of vegetation arrangement, and based on a thinning harvest where part of densely mixed vegetation is removed, under limited conditions where plowing, fertilizer, agricultural chemicals, or anything is not used except for seeds and seedlings (e.g., see PTL 1).

### CITATION LIST

### Patent Literature

[PTL 1] PCT Patent Publication No. WO2017/061281

### SUMMARY

### Technical Problems

According to the inventor of the present application, it has been confirmed that tea cultivated by synecoculture (registered trademark) exhibits more medicinal properties than tea cultivated by a conventional farming method.

Tea can be considered as a type of crude drug. It is therefore expected that, if crude drugs (plants producing crude drugs) other than tea are cultivated by synecoculture (registered trademark), the crude drugs to be obtained will also be rich in medicinal properties. Moreover, synecoculture (registered trademark) can be considered as a diversity-promoting cultivation method which promotes biodiversity and controls an ecosystem to produce plants. Accordingly, if crude drugs are cultivated by using the diversity-promoting cultivation method, the crude drugs to be obtained are expected to be rich in medicinal properties.

However, it is not yet known what cultivation condition is appropriate for cultivation of a particular crude drug, such as a crude drug desired by a user, using the diversity-promoting cultivation method like synecoculture (registered trademark), i.e., what cultivation condition is highly reproducible when a particular crude drug is cultivated by using the diversity-promoting cultivation method.

The present technology has been developed in consideration of the abovementioned circumstances, and the object thereof is to provide an appropriate cultivation condition for cultivating a crude drug by using a diversity-promoting cultivation method.

### Solution to Problems

According to the present technology, there is provided an information processing device or a program. The information processing device includes a first identification unit that identifies a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation. The program causes a computer to function as the information processing device.

According to the present technology, there is provided an information processing method including identifying a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.

According to the present technology, a cultivation condition for cultivating a specific crude drug is identified with use of a first model that associates a crude drug with a cultivation condition for cultivating the crude drug by a diversity-promoting cultivation method for promoting biodiversity and controlling an ecosystem to produce vegetation.

The information processing device may be either an independent device or an internal block included in one device.

In addition, the program may be provided by being transferred via a transfer medium or by being recorded in a recording medium.

### BRIEF DESCRIPTION OF DRAWINGS

FIG. 1 is a block diagram depicting a configuration example of an herbal medicine industry support system according to one embodiment to which the present technology is applied.

FIG. 2 is a diagram explaining a procedure for retrieving a cultivation condition for cultivating a medicinal plant by using synecoculture (registered trademark), and obtaining a generative prescription for producing, with the use of a crude drug obtained from the medicinal plant, an herbal medicine which increases health benefits.

FIG. 3 is a diagram explaining allelopathy.

FIG. 4 is a diagram depicting an example of phytochemicals that increase through an interaction with an insect.

FIG. 5 is a diagram depicting an example for presenting interactions between creatures which are related to a crude drug.

FIG. 6 is a diagram depicting a result of metabolomic analysis of tea (coarse tea) cultivated by synecoculture (registered trademark) and tea cultivated by a conventional farming method.

FIG. 7 is a diagram explaining an outline of a crude drug flower model associating crude drugs (medicinal plants producing the crude drugs) with cultivation conditions for cultivating the crude drugs.

FIG. 8 is a diagram explaining construction of the crude drug flower model.

FIG. 9 is a diagram depicting an example of construction of a flower model of a desired crude drug.

FIG. 10 is a diagram depicting another example of the construction of the flower model of the desired crude drug.

FIG. 11 is a diagram depicting an example of a manual for quality management to ensure the quality of a crude drug.

FIG. 12 is a diagram depicting an example of HPLC (High Performance Liquid Chromatography) patterns of alkaloid contained in Uncaria Hook which is a type of crude drug.

FIG. 13 is a diagram depicting an example of a quantity of ingredients contained in Uncaria Hook harvested in each of farm fields in production areas.

FIG. 14 is a diagram explaining FIM (Functional Independence Measure) as an example of indexes of health benefits.

FIG. 15 is a diagram depicting a result of an experiment concerning an FIM improvement as health benefits resulting from ingestion of tea cultivated by synecoculture (registered trademark).

FIG. 16 is a diagram explaining an outline of a health benefit flower model which associates health benefits with herbal medicines.

FIG. 17 is a diagram depicting an example of health parameters other than an herbal medicine.

FIG. 18 is a diagram depicting an example of construction of a flower model of a desired health benefit.

FIG. 19 is a diagram explaining blending of crude drugs to produce an herbal medicine.

FIG. 20 is a diagram depicting an example in which a crude drug blending quantity is calculated by using linear programming such that beneficial ingredients are equal to or above a reference value and toxic ingredients are equal to or below a reference value and such that an objective function representing a change of a desired health benefit is maximized.

FIG. 21 is a diagram depicting an example in which a crude drug blending quantity is calculated by using non-linear programming such that beneficial ingredients are equal to or above a reference value and toxic ingredients are equal to or below a reference value and such that the objective function representing a change of a desired health benefit is maximized.

FIG. 22 is a diagram depicting crude drugs blended to produce herbal medicines that are classified into a pungent-warm exterior-releasing medicine.

FIG. 23 is a diagram depicting an example of a generative prescription.

FIG. 24 is a diagram explaining a framework of dynamic real-time management of a super-diversity management system.

FIG. 25 is a block diagram depicting a functional configuration example of a server 13.

FIG. 26 is a diagram explaining an outline of construction of a crude drug flower model which is performed by a crude drug flower model construction unit 22.

FIG. 27 is a diagram explaining an outline of calculation of a crude drug blending quantity which is performed by a blending quantity calculation unit 24.

FIG. 28 is a flowchart explaining an example of a process of identifying a cultivation condition by the crude drug flower model construction unit 22.

FIG. 29 is a flowchart explaining an example of a process of identifying a health factor by a health benefit flower model construction unit 23.

FIG. 30 is a flowchart explaining an example of a process of calculating a crude drug blending quantity by the blending quantity calculation unit 24.

FIG. 31 is a block diagram depicting a configuration example of a computer according to one embodiment to which the present technology is applied.

### DESCRIPTION OF EMBODIMENT

### <Herbal Medicine Industry Support System According to One Embodiment>

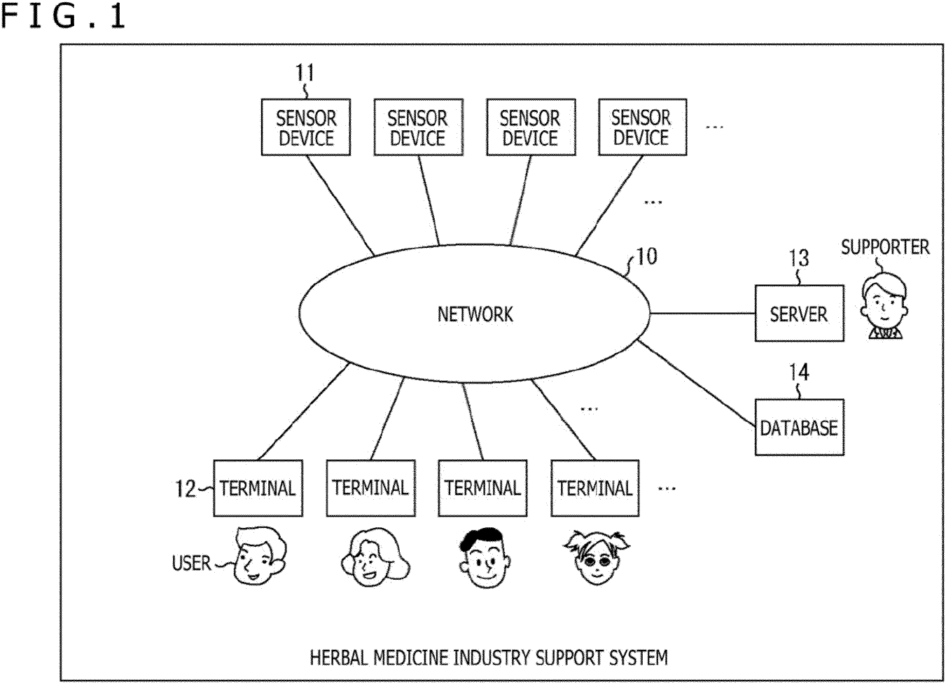

FIG. 1 is a block diagram depicting a configuration example of an herbal medicine industry support system according to one embodiment to which the present technology is applied.

The herbal medicine industry support system in FIG. 1 includes a network 10, one or more sensor devices 11, one or more terminals 12, a server 13, and a database 14.

The herbal medicine industry support system collects big data which includes various types of data (information) obtained by observing an ecosystem including a farm field or the like where a crude drug (a medicinal plant producing the crude drug) used to produce an herbal medicine is cultivated, data obtained by analyzing a crude drug cultivated in a farm field, clinical data regarding a person having ingested an herbal medicine, and other various types of data.

Thereafter, the herbal medicine industry support system obtains information for supporting an herbal medicine industry, on the basis of the big data, and supplies the information to a user or the like.

The sensor devices 11, the terminals 12, the server 13, and the database 14 are connected to the network 10 in a wired or wireless manner to communicate with each other.

The sensor device 11 includes a sensor for sensing various types of physical quantities, and has a communication function for transmitting sensor data (data indicating sensed physical quantities) obtained by the sensor as a result of the sensing. Moreover, if necessary, the sensor device 11 has a position detection function for detecting its own position by using GPS (Global Positioning System) or the like, for example.

The sensor device 11 senses physical quantities by using the sensor. Further, the sensor device 11 transmits the sensor data obtained by the sensing to the database 14 via the network 10 by using the communication function. The sensor data is transmitted from the sensor device 11 to the database 14 together with position information indicating a position of the sensor device 11 detected by the position detection function of the sensor device 11, as necessary.

For example, a sensor which senses electromagnetic waves including light, such as a sensor which captures images by sensing light (image sensor), and a sensor which senses sound (microphone) are adoptable as the sensor included in the sensor device 11. Further, a sensor which senses physical quantities such as temperature, humidity, humidity, geomagnetism, atmospheric pressure, and smells as various types of environmental information is also adoptable as the sensor included in the sensor device 11, for example.

The sensor device 11 is disposed in a farm field or the like where a crude drug is cultivated. The sensor device 11 can manually be disposed at a predetermined position. Alternatively, the sensor device 11 may be scattered over the farm field from a moving airplane, vessel, automobile, or the like.

For example, the sensor device 11 senses images of plants, bugs, or the like, acoustics such as a sound of wind, a sound of bugs, and a sound of rustling leaves, temperature such as air temperature and soil temperature, humidity, geomagnetism, and the like in the farm field (and surroundings of the farm field). Thereafter, the sensor device 11 transmits the sensor data obtained by the sensing to the database 14 via the network 10.

The terminal 12 is an information processing device used by a user who gains support from the herbal medicine industry or a user who supports the herbal medicine industry. For example, a portable terminal such as a smartphone, a tablet, and a wearable terminal is adoptable as the terminal 12. In addition, for example, a notebook PC (Personal Computer), a desktop PC, and a device having both a communication function and an input/output function (interface) for inputting and outputting information from and to the user are adoptable as the terminal 12.

For example, the user who gains support from the herbal medicine industry or the user who supports the herbal medicine industry is a person who cultivates a crude drug used to produce an herbal medicine (this person hereinafter includes a corporate body and an organization where appropriate), a person who produces an herbal medicine by blending crude drugs (this person includes a person who prescribes an herbal medicine), a person who ingests an herbal medicine, a person in charge of clinical trials or nursing of the person ingesting the herbal medicine, or others.

For example, the terminal 12 transmits various types of data to the database 14 via the network 10 according to an operation performed by the user.

For example, a person cultivating a crude drug carries out observation at various places in an environment where the crude drug is cultivated, such as a farm field, by using the terminal 12, and transmits observation values indicating a result of the observation to the database 14 via the network 10.

Moreover, for example, a person ingesting an herbal medicine or a person in charge of clinical trials of the person ingesting the herbal medicine uses the terminal 12 to transmit the ingested herbal medicine, a lifestyle of the person ingesting the herbal medicine, data (observation values) of the clinical trials, and the like to the database 14 via the network 10.

Further, the terminal 12 receives various types of data transmitted (supplied) from the server 13 via the network 10, and presents the received data to the user by displaying the data as images or outputting the data as sounds.

For example, the terminal 12 of a person cultivating a crude drug can receive, from the server 13, a cultivation condition as a cultivation method for cultivating the crude drug by synecoculture (registered trademark) or the like, and display the received cultivation condition.

Moreover, for example, the terminal 12 of a person who produces an herbal medicine by blending crude drugs can receive, from the server 13, a blending quantity of the crude drugs used to produce the herbal medicine, and display the received blending quantity.

Further, for example, the terminal 12 of a person ingesting an herbal medicine or a person in charge of clinical trials or nursing of the person ingesting the herbal medicine can receive, from the server 13, information associated with an herbal medicine which offers a health benefit desired by the person ingesting the herbal medicine, and display the received information.

The server 13 is an information processing device managed by a supporter who supports the herbal medicine industry.

The server 13 uses data registered in the database 14 to obtain information for supporting the herbal medicine industry, such as a cultivation condition for cultivating a specific crude drug by synecoculture (registered trademark) or the like, a quantity of crude drugs used to produce a specific herbal medicine, and information associated with an herbal medicine offering a specific health benefit. Thereafter, the server 13 transmits the cultivation condition, the blending quantity, and the information associated with the herbal medicine described above to the terminal 12 via the network 10, and thus, the abovementioned information is supplied to the terminal 12.

The database 14 registers (stores) data (information) transmitted from the terminal 12 via the network 10.

Note that the server 13 may be either a single server or a set of multiple servers. Moreover, the database 14 includes not only a database where data received from the terminal 12 is registered, but also a database where data necessary for supporting the herbal medicine industry, such as required reference values of beneficial ingredients and toxic ingredients included in an herbal medicine produced according to a classic prescription, is registered.

### <Procedure for Obtaining Generative Prescription to Produce Herbal Medicine>

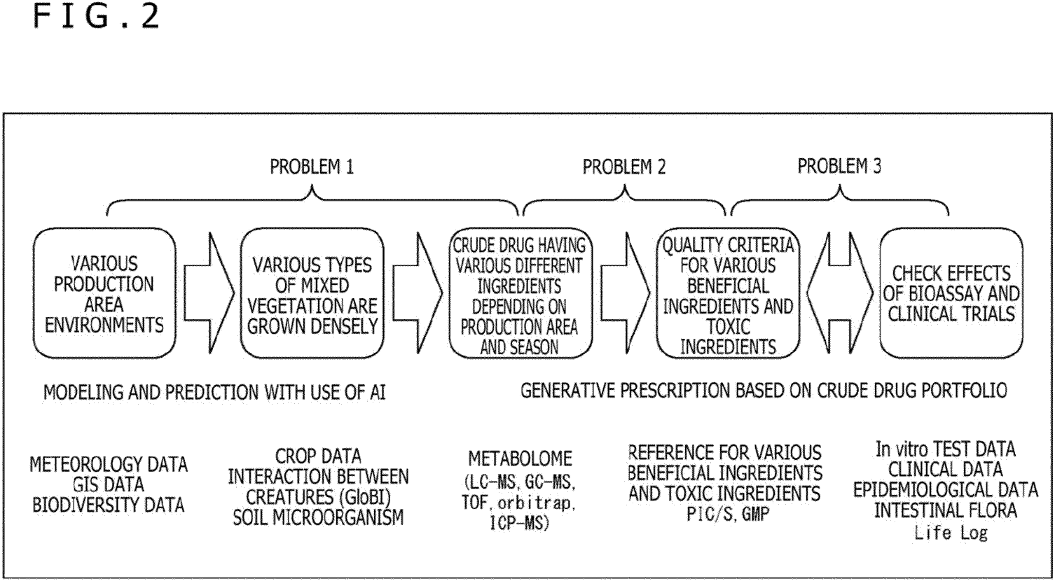

FIG. 2 is a diagram explaining a procedure for retrieving a cultivation condition for cultivating a medicinal plant by using synecoculture (registered trademark), and obtaining a generative prescription for producing, with the use of a crude drug obtained from the medicinal plant, an herbal medicine which increases health benefits.

Cultivation of a medicinal plant based on synecoculture (registered trademark) is conducted in various production area environments where various types of mixed vegetation are grown densely. In addition, a crude drug obtained from the medicinal plant has multiple different ingredients depending on the production area, season, or the like. In such cultivation of a medicinal plant based on synecoculture (registered trademark), there is a problem in retrieval of information regarding how to cultivate a medicinal plant producing a desired crude drug (containing desired ingredients), i.e., a cultivation method for cultivating a medicinal plant producing a desired crude drug (method for cultivating densely mixed vegetation) (problem 1).

According to the present technology, a cultivation condition is retrieved as a cultivation method for cultivating a desired medicinal plant (producing a desired crude drug), by performing modeling (learning) and prediction with the use of AI (Artificial Intelligence) on the basis of various types of data corresponding to a cultivation condition used for cultivation using synecoculture (registered trademark) or the like, and on the basis of a result of metabolomic analysis of a crude drug obtained from a medicinal plant cultivated under the various cultivation conditions.

Examples of the various types of data corresponding to the cultivation conditions concerning the various production environments include meteorology data, GIS (Geographic Information System) data, and biodiversity data (e.g., information associated with creatures present in the production environments). Example of the various types of data corresponding to the cultivation conditions concerning various types of mixed vegetation densely grown include crop data (e.g., information associated with cultivated plants (crops)), data regarding interactions between creatures (GloBI (Global Biotic Interactions)), and data regarding soil microorganisms contained in soil where plants are cultivated.

In addition, a crude drug obtained from a medicinal plant has multiple different ingredients depending on the production area, season, or the like. Moreover, an herbal medicine formulated by using such a crude drug is required to meet quality criteria for multiple beneficial ingredients and toxic ingredients (toxic substances). Accordingly, it leaves a problem in terms of how to formulate such an herbal medicine (problem 2).

According to the present technology, for example, prescription of an herbal medicine meeting quality criteria for multiple beneficial ingredients and toxic ingredients is retrieved by using a result of metabolomic analysis of a crude drug or the like, according to PIC/S (Pharmaceutical Inspection Convention and Pharmaceutical Inspection Cooperation Scheme), GMP (Good Manufacturing Practice), or the like. The metabolomic analysis of a crude drug can be performed by using various types of mass spectrometers such as LC-MS, GC-MS, TOF, orbitrap, and ICP-MS.

Also, there is problem in an evaluation method of health benefits offered by an herbal medicine formulated by using a crude drug obtained from a medicinal plant, i.e., how to evaluate health benefits of an herbal medicine (problem 3).

According to the present technology, effects of a bioassay and clinical trials on a person who ingests the herbal medicine and a person who does not ingest the herbal medicine are checked, and health benefits of an herbal medicine are thus evaluated. The effects of the bioassay and clinical trials can be checked by using In vitro test data, clinical data, epidemiological data, data regarding intestinal flora, lifelog, or the like.

By feeding back the (evaluation results of) health benefits of an herbal medicine to an herbal prescription, it becomes possible to obtain a crude drug portfolio for formulating such an herbal medicine that is capable of offering a desired health benefit and that meets quality criteria for multiple beneficial ingredients and toxic ingredients, and therefore, the herbal medicine can be formulated (generated) according to the obtained crude drug portfolio.

### <Allelopathy>

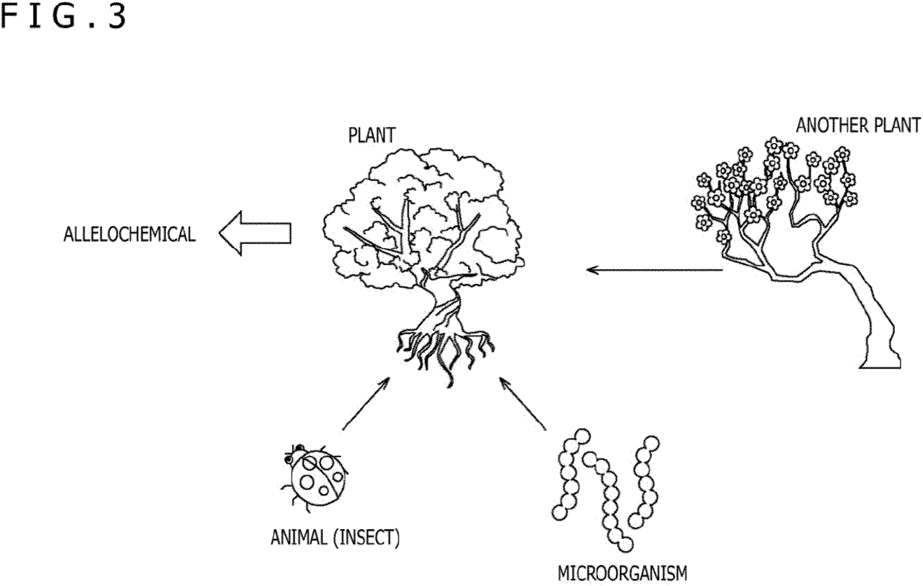

FIG. 3 is a diagram explaining allelopathy.

A plant interacts with the surrounding creatures in various ways such as promoting or suppressing the growth of the surrounding creatures, according to the surrounding other plants, animals (insects), microorganisms, and environmental stress. Such an interaction is called allelopathy. Allelopathy generates chemical substances called allelochemicals which constitute bioactive compounds. Allelochemicals are mainly generated as secondary metabolic products. Secondary metabolic products of plants have medicinal effects (medicinal benefits). For example, plants produce and release phytochemicals (plant chemical substances) when insects bite the plants. The phytochemicals have medicinal effects.

In a conventional farming method using monoculture, for example, chemical fertilizers or agricultural chemicals are used to promote the growth of plants on the ground. In such a case, however, the number of microorganisms in the soil is decreased due to the chemical fertilizers and the agricultural chemicals, and an ecosystem of the soil is destroyed. Consequently, biodiversity is lost, and substances (bioactive compounds) (pharmaceutical substances) that are contained in plants and that have medicinal effects produced by interactions with microorganisms or the like in the soil decrease.

Accordingly, it is assumed in the present technology that cultivation of medicinal plants is performed by using not a conventional farming method but a diversity-promoting cultivation method such as synecoculture (registered trademark).

The diversity-promoting cultivation method is a cultivation method for promoting biodiversity and controlling an ecosystem to produce plants. Synecoculture (registered trademark) is an open-field crop cultivation method in which plowing, fertilizer, agricultural chemicals, or anything is not used except for seeds and seedlings. In synecoculture, the ecosystem is constructed and controlled by utilizing characteristics of plants, to produce useful plants in an ecological optimal state (ecological optimization). Synecoculture (registered trademark) is a type of diversity-promoting cultivation method.

Ecological optimization is a state where multiple species achieve their maximum growth while competing against and living symbiotically with each other, to the extent possible in given environmental conditions. On the other hand, the conventional farming method relies on physiological optimization which generally changes environmental conditions in order to optimize a single type of growth.

Synecoculture (registered trademark) can enrich biodiversity and enhance various ecosystem functions. The ecosystem functions herein refer to functions of adjusting environmental conditions such as temperature, humidity, a quantity of solar radiation, and organic matters and minerals in the soil, in such a manner that more creatures can live comfortably. With the enhanced ecosystem functions, it is possible to achieve a richer biodiversity. Accordingly, biodiversity and the ecosystem functions are enhanced together in a synergistic manner.

According to synecoculture (registered trademark), interactions increase in various ways as biodiversity becomes richer. Accordingly, crude drugs (medicinal plants producing the crude drugs) containing more bioactive compounds can be cultivated. Moreover, high-quality herbal medicines can be produced by using such crude drugs as described above. A case where the present technology is applied to synecoculture (registered trademark) will be described below. However, the present technology is also applicable to diversity-promoting cultivation methods other than synecoculture (registered trademark), such as a diversity-promoting cultivation method which involves plowing or uses fertilizers or agricultural chemicals that do not damage desired crude drugs.

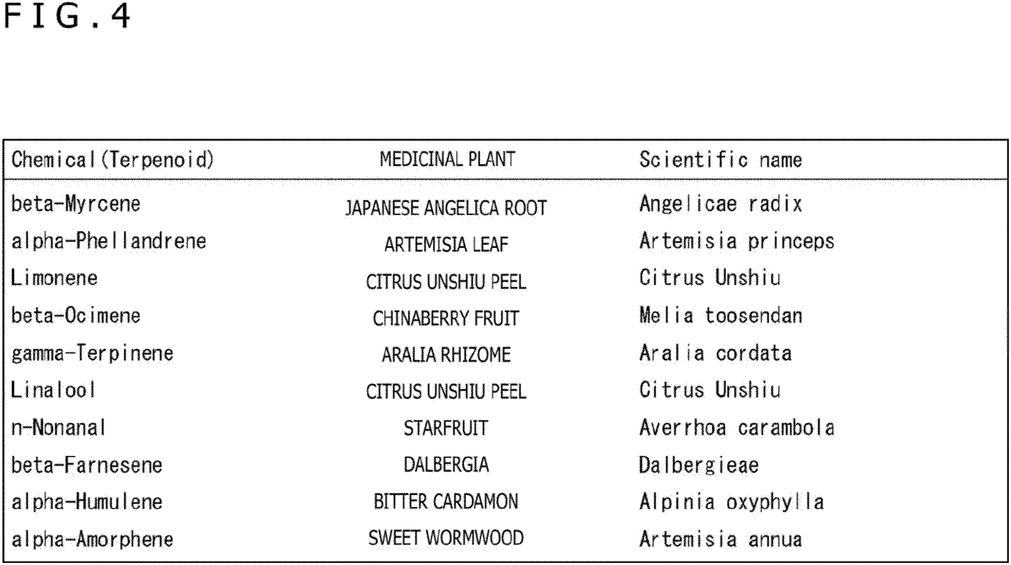

FIG. 4 is a diagram depicting an example of phytochemicals that increase through an interaction with an insect.

In FIG. 4, the second column from the left indicates a crude drug containing a phytochemical increased through an interaction with an insect, while the first column from the left indicates a phytochemical as a beneficial ingredient contained in the crude drug in the second column from the left. The third column from the left indicates a Latin scientific name of the phytochemical in the first column from the left.

Note that the crude drug in the second column from the left contains not only the one beneficial ingredient indicated in the first column from the left, but also multiple beneficial ingredients. In the first column from the left in FIG. 4, a typical beneficial ingredient of the crude drug is indicated.

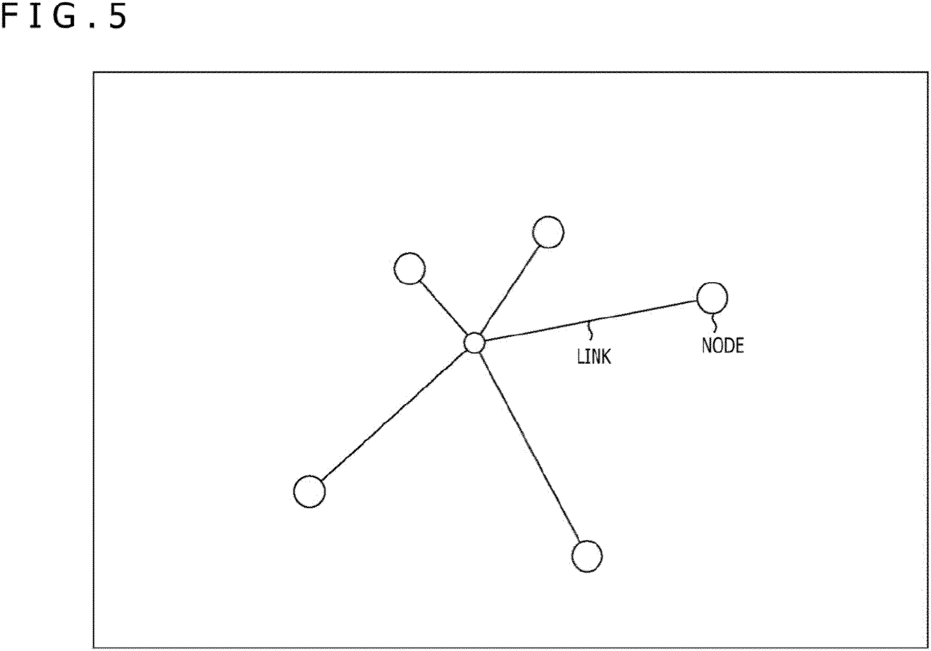

FIG. 5 is a diagram depicting an example for presenting interactions between creatures which are related to a crude drug.

The interactions between creatures which are related to a crude drug can be represented by a network (graph) including nodes and links. Each of the nodes represents a creature (species), and an interaction between creatures illustrated as the nodes is represented by the link.

In the network representing the interactions between creatures which are related to a crude drug, a node representing a crude drug (a medicinal plant producing the crude drug) and a node representing a creature having an interaction with the crude drug are connected to each other by a link representing the interaction. A degree of the interaction between the creatures represented by the nodes may be expressed by a length, a thickness, or the like of the link connecting the nodes representing the creatures.

For example, the network representing the interactions between creatures which are related to a crude drug may be constructed by using a data set provided by GloBI (Global Biotic Interactions), for example.

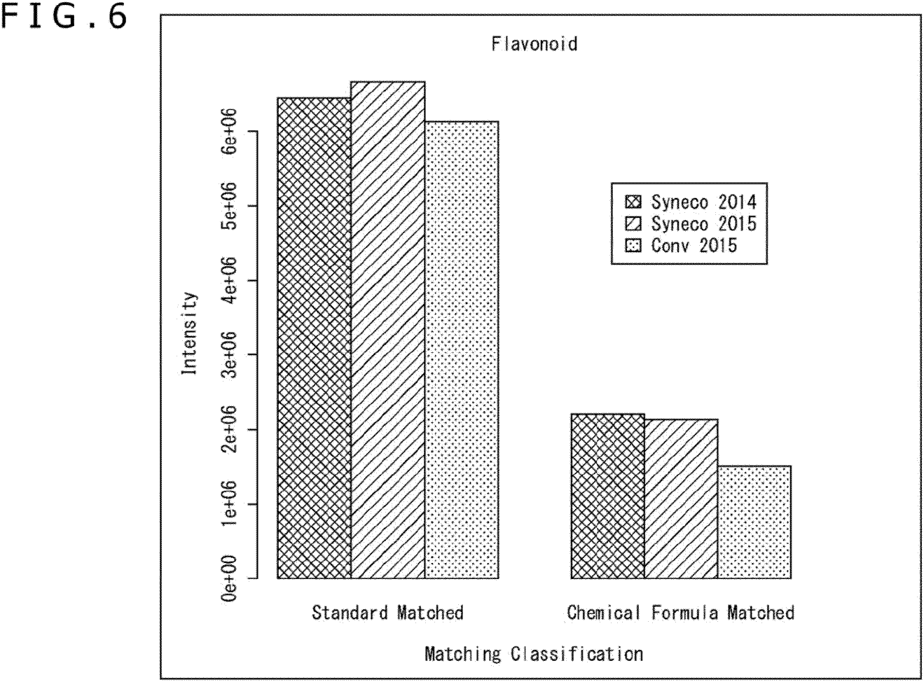

FIG. 6 is a diagram depicting a result of metabolomic analysis of tea (coarse tea) cultivated by synecoculture (registered trademark) and tea cultivated by a conventional farming method.

Specifically, FIG. 6 illustrates a flavonoid content of tea cultivated by synecoculture (registered trademark) and a flavonoid content of tea cultivated by the conventional farming method.

FIG. 6 illustrates a flavonoid content of tea (Syneco2014) cultivated by synecoculture (registered trademark) in 2014, a flavonoid content of tea (Syneco2015) cultivated by synecoculture (registered trademark) in 2015, and a flavonoid content of tea (Conv2015) cultivated by the conventional farming method in 2015.

A flavonoid content is calculated by identifying a chemical substance contained in tea, and multiplying intensity of the chemical substance identified as a flavonoid. Identification of the flavonoid is performed on the basis of a chemical formula level (Chemical Formula Matched) and a structure isomer level (Standard Matched).

As can be confirmed from FIG. 6, the tea cultivated by synecoculture (registered trademark) has a larger flavonoid content than the tea cultivated by the conventional farming method.

Note that, according to metabolomic analysis, the tea cultivated by synecoculture (registered trademark) exhibits approximately 200 types of different ingredients in comparison with the tea cultivated by the conventional farming method, and it is also confirmed that many of these ingredients of the tea cultivated by synecoculture are registered as medicinal properties.

Tea is a type of crude drug. Accordingly, when a crude drug is cultivated by synecoculture (registered trademark) in an environment where mixed plants are grown densely to promote interactions between creatures, the crude drug is expected to contain many medicinal ingredients (many types and quantities of medicinal ingredients).

According to the present technology, multiomics analysis is performed to evaluate which ingredient of the crude drug increases and which environment and combination of plants contribute to the increase of the ingredient, and a cultivation condition for cultivating a crude drug (desired crude drug) containing desired ingredients is identified by using a flower model (a crude drug flower model) described below.

Moreover, according to the present technology, when crude drugs cultivated by synecoculture (registered trademark) are blended to produce an herbal medicine, such a crude drug blending quantity that increase a desired health benefit while maintaining multiple types of beneficial ingredients equal to or above a reference value and multiple types of toxic ingredients equal to or below a reference value is calculated by using linear programming or non-linear programming. Thereafter, the crude drugs are blended according to the blending quantity thus calculated, to produce the herbal medicine.

Further, according to the present technology, an herbal medicine corresponding to a factor (health factor) offering a desired health benefit is identified by using a flower model described below (health benefit flower model).

The relation between the desired health benefit and the herbal medicine identified as the factor offering the desired health benefit is fed back to a production (formulation) process of the herbal medicine. Thereafter, upon the production (formulation) of the herbal medicine, a crude drug blending quantity is calculated such that an objective function is maximized (the desired health benefit is maximized). The objective function is obtained from the relation between the herbal medicine and the desired health benefit and represents a change (level) of the desired health benefit with respect to the herbal medicine (the crude drug contained in the herbal medicine).

The identification of the herbal medicine corresponding to the factor offering the desired health benefit, the feedback of the relation between the herbal medicine and the desired health benefit, and the calculation of the crude drug blending quantity which maximizes the objective function obtained from the relation between the herbal medicine and the desired health benefit are repeatedly carried out. In such a manner, accuracy of the identification of the herbal medicine corresponding to the factor offering the desired health benefit and accuracy of the calculation of the crude drug blending quantity which maximizes the objective function representing a change of the desired health benefit are improved.

### <Crude Drug Flower Model>

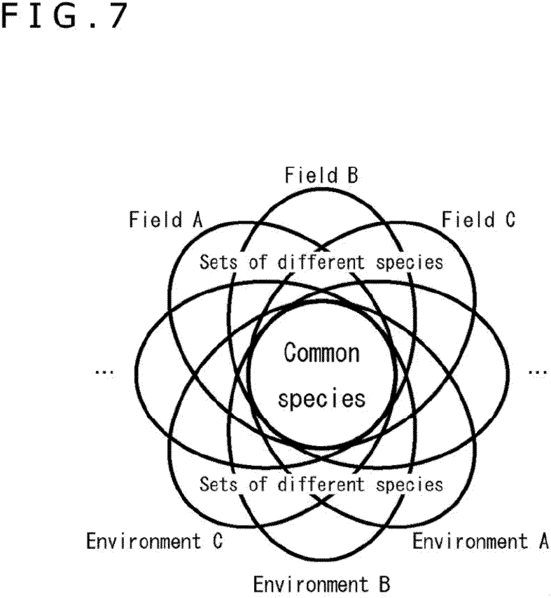

FIG. 7 is a diagram explaining an outline of a flower model of crude drugs (medicinal plants producing the crude drugs). In the flower model, the crude drugs are associated with cultivation conditions for cultivating the crude drugs.

In FIG. 7, respective dots represent various species of crude drugs (Set of different species), while ellipses each represent a cultivation condition for cultivating the corresponding crude drug. A dot within the ellipse represents a crude drug (species) that is cultivated (grown) only under the cultivation condition represented by the ellipse. The cultivation condition represented by each of the ellipses is an essential condition for cultivating the crude drug represented by the dot within the corresponding ellipse.

According to the present technology, through cultivation of various crude drugs using synecoculture (registered trademark), big data regarding various parameters associated with (presumed to be associated with) the cultivation of the crude drugs (hereinafter also referred to as cultivation parameters) is collected. Thereafter, by learning of the big data regarding the cultivation parameters of the various crude drugs with the use of AI (Artificial Intelligence), a cultivation parameter (type and value of the cultivation parameter) significant for cultivation of a crude drug is retrieved as a cultivation condition of the crude drug.

The relation between a crude drug and its cultivation condition is expressed in a form in which a dot (a region including the dot) representing the crude drug is included in an ellipse representing the condition for cultivation of the crude drug. This form resembles a flower shape which has ellipses as petals. Accordingly, a model expressed in this form is referred to as a flower model in the present embodiment.

In a crude drug flower model (first model), i.e., a flower model associating crude drugs with cultivation conditions, each petal can also be considered as a set of crude drugs (dots representing the crude drugs) that need to be cultivated in a cultivation condition represented by the corresponding petal (ellipse). In such a case, it can be said that the flower model includes petals each representing a set of crude drugs that need to be cultivated in a certain cultivation condition.

When the crude drug flower model, i.e., the flower model associating crude drugs and their cultivation conditions, is constructed, a petal (an ellipse as the petal) representing a cultivation parameter which may constitute a cultivation condition can be set (added) in an appropriate manner in learning of big data regarding various cultivation parameters. If a cultivation parameter represented by a petal constitute a significant cultivation condition for crude drug cultivation, the petal representing the cultivation condition changes in such a manner as to include a dot representing a crude drug that need to be cultivated in the cultivation condition. On the other hand, if the cultivation parameter represented by the petal does not constitute a significant cultivation condition, the corresponding petal disappears.

Note that cultivation parameters that are obviously required for cultivation of all crude drugs, such as the presence or absence of air in a case of cultivating crude drugs on the earth where air is always present, can be excluded from (setting of) the flower model. On the other hand, in a case where crude drugs are cultivated on the earth where air is present and on the moon where air is absent, for example, the presence or absence of air and compositions thereof can be considered (set) as cultivation parameters.

According to the crude drug flower model, for example, an ecological niche for a desired crude drug such as a crude drug having more beneficial ingredients (a crude drug containing beneficial ingredients of a predetermined value or more), i.e., a cultivation condition used as an appropriate cultivation method for cultivating the crude drug, can be identified.

For example, the flower model of the desired crude drug can be constructed by retrieving a cultivation condition which is represented by a petal including a dot representing the desired crude drug, by using a gradient method.

According to the present technology, cultivation parameters including at least a parameter associated with synecoculture (registered trademark) are set as cultivation parameters which may constitute cultivation conditions represented by petals. Thereafter, a cultivation condition (a cultivation parameter constituting the cultivation condition) represented by a petal including a dot representing a desired crude drug is retrieved by a gradient method to construct a flower model of a desired crude drug. The flower model is a flower model which associates the desired crude drug with a cultivation condition for cultivating the desired crude drug by synecoculture (registered trademark). Thereafter, according to the present technology, the flower model is used to identify a cultivation condition (cultivation method) for cultivating the desired crude drug by synecoculture (registered trademark), such as a cultivation condition for promoting biodiversity and interactions to increase beneficial ingredients of the crude drug. With the use of the cultivation condition identified by the present technology to cultivate a desired crude drug, the desired crude drug is cultivated by synecoculture (registered trademark) with higher reproducibility.

For example, the parameters associated with synecoculture (registered trademark), i.e., the cultivation parameters which may become the cultivation conditions for cultivating the crude drugs by synecoculture (registered trademark), include information regarding a local quantity of solar radiation, diversity of soil microorganisms, types of mixed plants, a height of ridges, a soil quality (e.g., amount of water and drainage in the soil), and the like.

Note that, in a case of cultivating a crude drug by using the conventional farming method, parameters associated with the conventional farming method, such as information indicating plowing, fertilization, use of agricultural chemicals, and an amount of sprinkling, are set as cultivation parameters.

In a case of cultivating a crude drug represented by a certain dot in the crude drug flower model, cultivation conditions represented by all petals each including the dot are applied cumulatively (by logical conjunction).

For example, according to the flower model depicted in FIG. 7, a range indicated by Common species is included in petals representing conditions of farm fields A, B, and C, conditions of environments (Environments) A, B, and C, and other conditions, which are cultivation conditions. Accordingly, the conditions of the farm fields A, B, and C, the conditions of the environments A, B, and C, and other conditions are all applied to the cultivation conditions of the crude drugs included in the range indicated by Common species.

For example, each of the conditions of the farm fields indicates information associated with the corresponding farm field, such as mixed plants growing in the farm field. For example, each of the conditions of the environments indicates information associated with an environment where the crude drug is cultivated, such as a soil quality, a sunny place, and a shade.

According to the crude drug flower model, it is possible to identify not only a cultivation condition for cultivating a desired crude drug, but also a crude drug that is able to (suitable to) be cultivated in a current environment (cultivation condition) by setting a petal indicating the current environment. With this, for example, in a case of a change of the climate in the farm field, a crude drug suitable for cultivation in the farm field after the change of the climate is predictable.

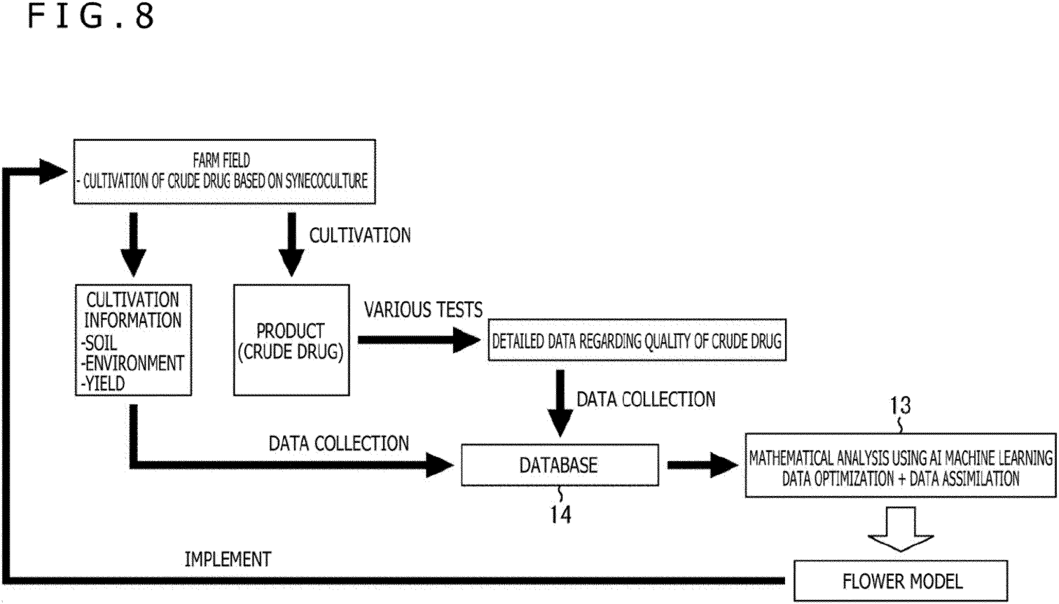

FIG. 8 is a diagram explaining construction of the crude drug flower model.

Cultivation of crude drugs based on synecoculture (registered trademark) is conducted in various farm fields. In addition, for example, a user in a farm field uses the terminal 12 (FIG. 1) to collect cultivation information (information which may constitute cultivation parameters) associated with crude drug cultivation based on synecoculture (registered trademark), such as data associated with the soil, the environment, the yield of products including the crude drug harvested in the farm field, or the like, and registers the collected cultivation information in the database 14. Sensor data obtained as a result of the sensing by the sensor device 11 disposed in the farm field is also registered in the database 14.

Moreover, concerning crude drugs harvested as products in the farm field, various types of tests such as metabolomic analysis are executed, and detailed data associated with the quality of the crude drugs is collected. The data is also registered in the database 14.

The server 13 performs mathematical analysis for the data registered in the database 14 by use of AI machine learning, to achieve data optimization (weight saving) and data assimilation. Moreover, in the server 13, significant data resulting from the abovementioned processing and associated with cultivation of the crude drugs is used as cultivation parameters or the like to construct a flower model of a desired crude drug designated by the user operating the terminal 12, for example.

Thereafter, the server 13 uses the flower model of the desired crude drug to identify a cultivation condition in synecoculture (registered trademark) as a cultivation method for cultivating the desired crude drug, and supplies (transmits) the identified cultivation condition to the terminal 12.

In the farm field, the user implements (embodies) the cultivation condition supplied from the server 13 to the terminal 12, and cultivates the crude drug by synecoculture (registered trademark). For example, the user in the farm field uses the terminal 12 to collect cultivation information associated with cultivation of the crude drug by synecoculture (registered trademark), and registers the collected cultivation information in the database 14.

Similar processing (work) is subsequently repeated. In such a manner, the desired crude drug can be cultivated by synecoculture (registered trademark) with higher reproducibility.

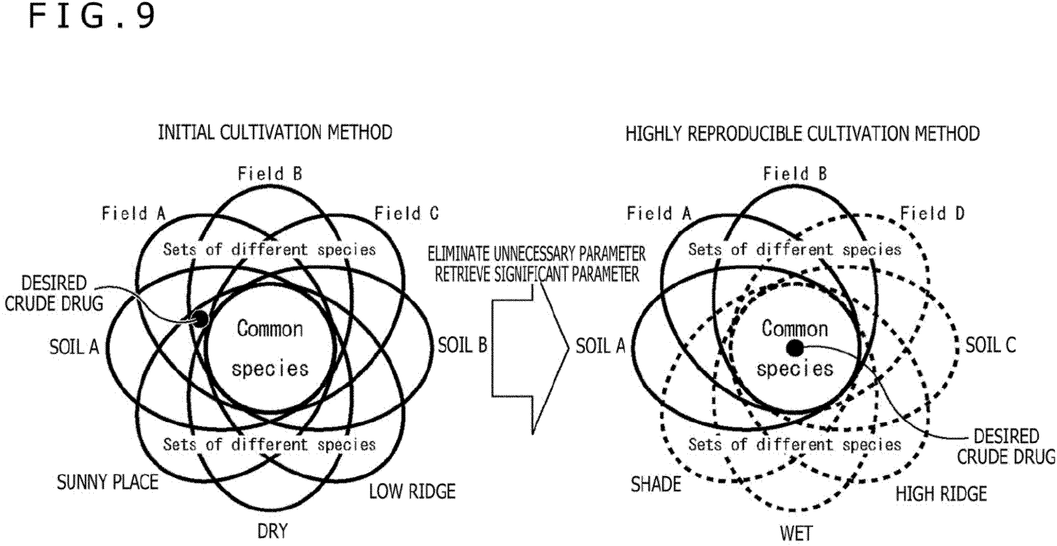

FIG. 9 is a diagram depicting an example of construction of a flower model of a desired crude drug.

A flower model on the left of FIG. 9 represents a flower model which includes petals (ellipses) each indicating a cultivation condition as a cultivation method in farm fields using synecoculture (registered trademark) where a desired crude drug is to be cultivated.

According to the left flower model, cultivation conditions in the farm field where the desired crude drug is to be cultivated include the conditions of the farm fields (Field) A, B, and C, a soil quality A, a soil quality B, a sunny place, drying, and low ridges.

In the left flower model, a dot representing the desired crude drug is located out of a range indicated by Common species where all cultivation conditions in the farm fields, in which the desired crude drug is to be cultivated, overlap with each other.

Upon the construction of the flower model of the desired crude drug, in order to cultivate the desired crude drug by synecoculture (registered trademark), unnecessary cultivation parameters are eliminated, and significant cultivation parameters are retrieved as conditions for cultivation of the desired crude drug. Thus, the flower model that includes petals (ellipses) each having the dot representing the desired crude drug is constructed.

A flower model on the right of FIG. 9 represents a flower model constructed by retrieval of a cultivation condition for cultivating the desired crude drug.

In the right flower model, the condition of the farm field C, the soil quality B, the sunny place, the drying, and the low ridges, which are the cultivation conditions included in the left flower model, are eliminated (disappear) since they are unnecessary cultivation parameters in the cultivation of the desired crude drug.

Moreover, in the right flower model, in addition to the conditions of the farm fields A and B and the soil quality A, which are the cultivation conditions included in the left flower model, a condition of a farm field D, a soil quality C, a shade, wetting, and high ridges, which are not included in the left flower model, are retrieved since they are significant cultivation parameters in the cultivation of the desired crude drug.

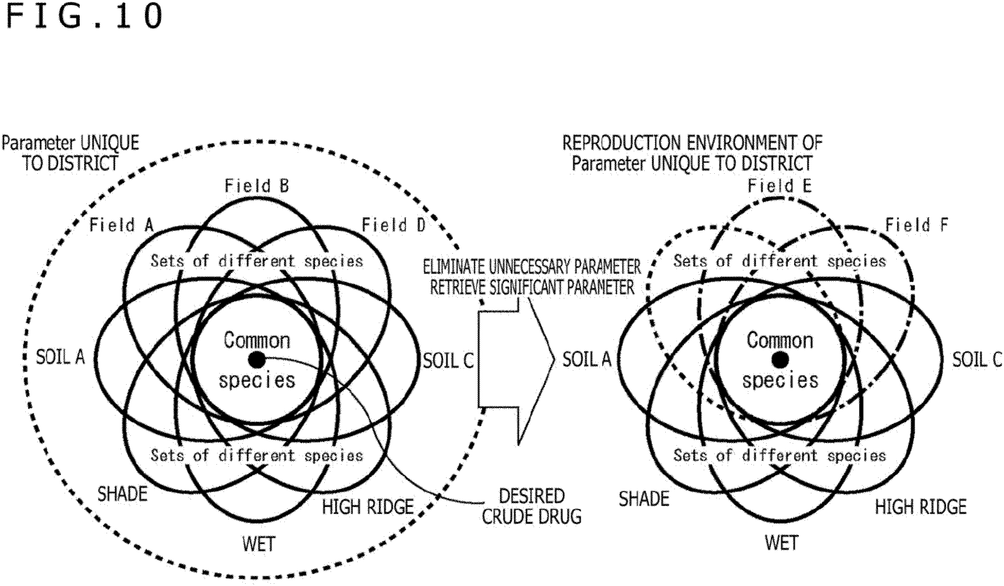

FIG. 10 is a diagram depicting another example of the construction of the flower model of the desired crude drug.

A flower model on the left of FIG. 10 is a flower model which associates a desired crude drug with its cultivation condition. The left flower model is constructed by using such data as cultivation information that is collected in a case where crude drugs are cultivated by synecoculture (registered trademark) in farm fields located only in a certain single district.

In the flower model constructed by using data collected in the farm fields located only in the single district, cultivation parameters that are unique to the single district affect all crude drugs cultivated in the farm fields in the single district, and are represented by petals including all dots representing the crude drugs cultivated in the farm fields in the single district, as indicated by a dotted circle.

A flower model on the right of FIG. 10 is a flower model which associates a desired crude drug with its cultivation condition. The right flower model is constructed by using such data as cultivation information that is collected in a case where crude drugs are cultivated by synecoculture (registered trademark) in farm fields located in multiple districts including, in addition to the single district, one or more other districts.

In the flower model constructed by using data collected in the farm fields located in the multiple districts, cultivation parameters that are unique to the single district may affect crude drugs cultivated in the farm fields in the single district, but do not necessarily affect crude drugs cultivated in the farm fields in the other districts. Accordingly, these cultivation parameters are represented by petals including only dots representing the affected crude drugs, as indicated by dotted ellipses.

### <Quality Assurance of Crude Drug>

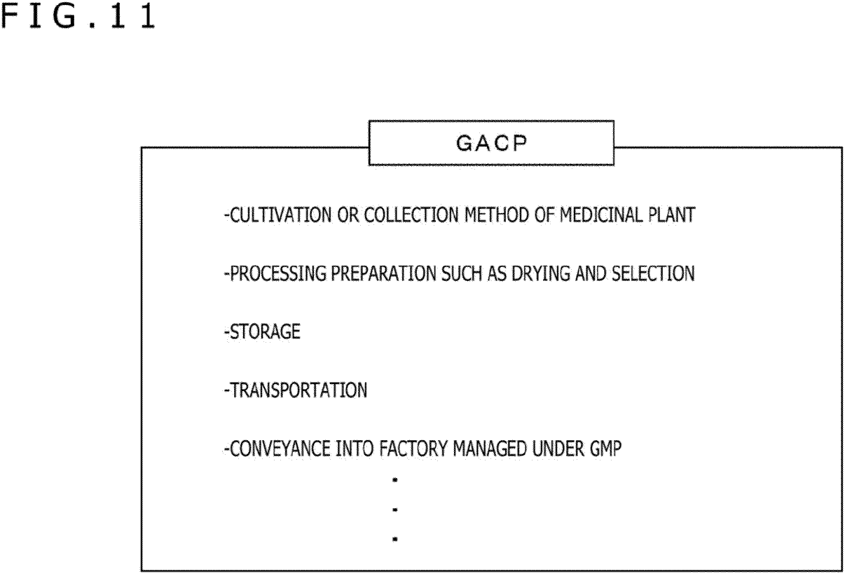

FIG. 11 is a diagram depicting an example of a manual for quality management to ensure the quality of a crude drug.

In order to ensure the quality of crude drugs obtained from medicinal plants cultivated in a farm field, GACP (Good Agricultural and Collection Practice: management of production process of raw materials for plant-based medical supplies (BRM)) as a manual for quality management is established. The medicinal plants are handled according to GACP mentioned herein.

GACP specifies matters associated with a cultivation and collection method of medicinal plants, processing preparation such as drying and selection of medicinal plants, storage of medicinal plants, transportation of medicinal plants, conveyance of medicinal plants into factories managed under GMP, and the like.

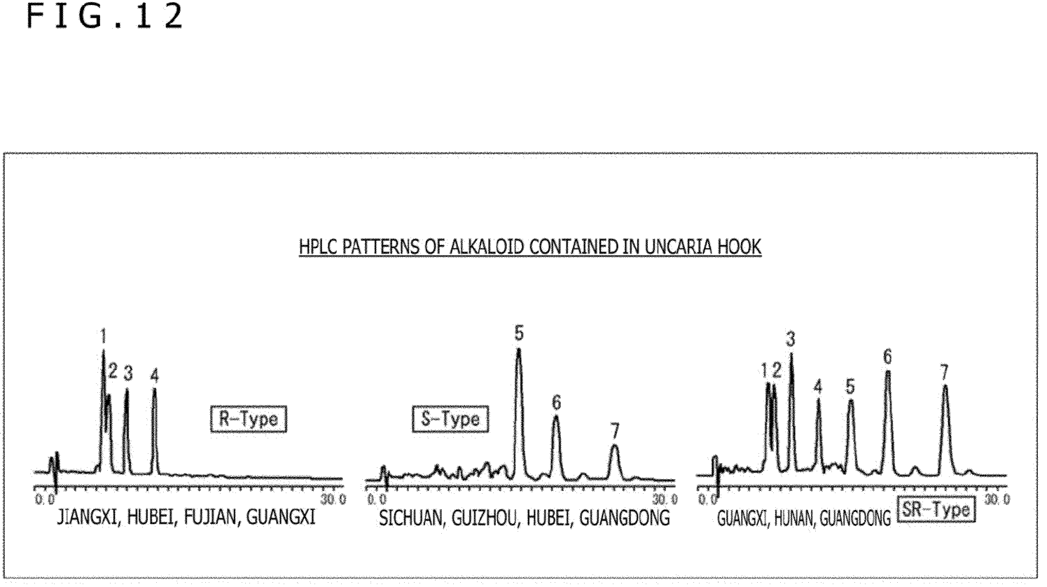

FIG. 12 is a diagram depicting an example of HPLC (High Performance Liquid Chromatography) patterns of alkaloid contained in Uncaria Hook which is a type of crude drug.

FIG. 12 illustrates HPLC patterns of alkaloid contained in Uncaria Hook produced in different production areas.

The HPLC patterns of alkaloid contained in Uncaria Hook differ between production areas of Uncaria Hook, and are classified into R-type, S-type, SR-type, SR2-type, and other patterns.

As described above, the HPLC patterns of alkaloid contained in Uncaria Hook differ between the production areas of Uncaria Hook. Accordingly, ingredient composition ratios of various types of ingredients contained in Uncaria Hook also differ between the production areas of Uncaria Hook.

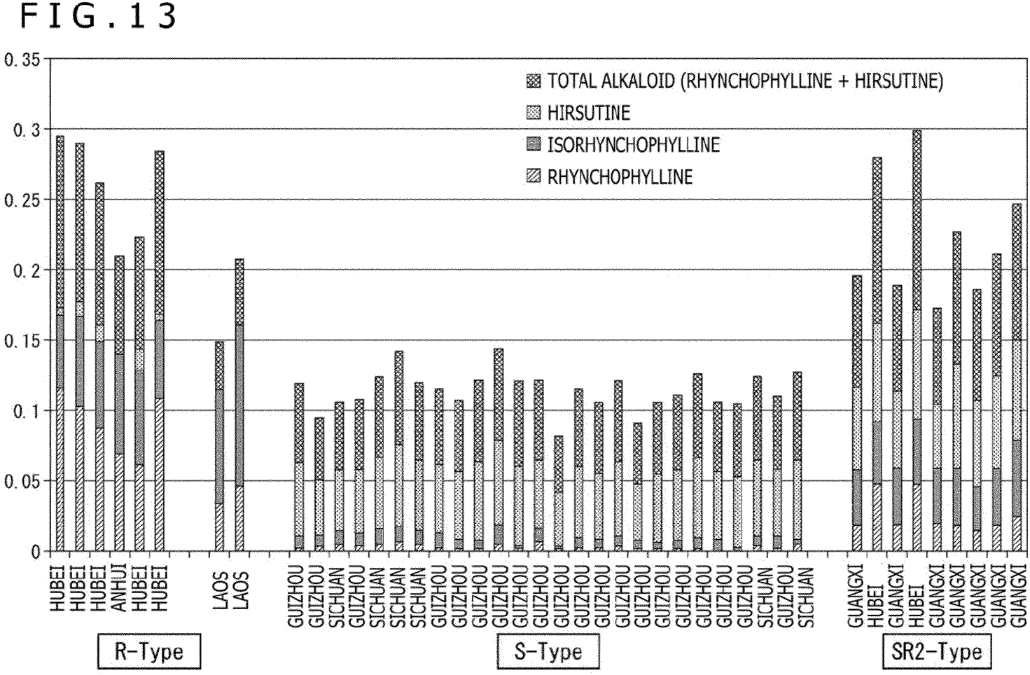

FIG. 13 is a diagram depicting an example of a quantity of ingredients contained in Uncaria Hook harvested in each of farm fields in production areas.

Uncaria Hook is used as an antispasmodic drug or an analgesic drug, and contains alkaloid such as rhynchophylline, isorhynchophylline, corynoxeine, hirsutine, and hirsuteine. Substances derived from Uncaria rhynchophylla contains substantially no hirsutine and no hirsuteine. As illustrated in FIG. 13, the quantities of the ingredients of Uncaria Hook differ between production areas.

As described above, the ingredient composition ratios and the quantities of the ingredients of Uncaria Hook differ between production areas. In other words, the quality of Uncaria Hook differs depending on the production area. Accordingly, even if Uncaria Hook is handled according to GACP as the manual explained with reference to FIG. 11, it is difficult to obtain Uncaria Hook having a desired quality, i.e., desired ingredients (desired type and quantity of ingredients).

In a case where an herbal medicine is to be produced by blending crude drugs including Uncaria Hook, in order to meet criteria for the herbal medicine, Uncaria Hook is empirically blended on a production-area basis, for example, to prepare Uncaria Hook having desired ingredients.

This is similarly applicable to crude drugs other than Uncaria Hook. In a case where an herbal medicine is to be produced by blending crude drugs having the same crude drug name but having different ingredient composition ratios or different quantities of ingredients, it is necessary to change a crude drug blending method, such as a crude drug blending quantity, according to the quality of crude drugs.

### <Health Benefit Flower Model>

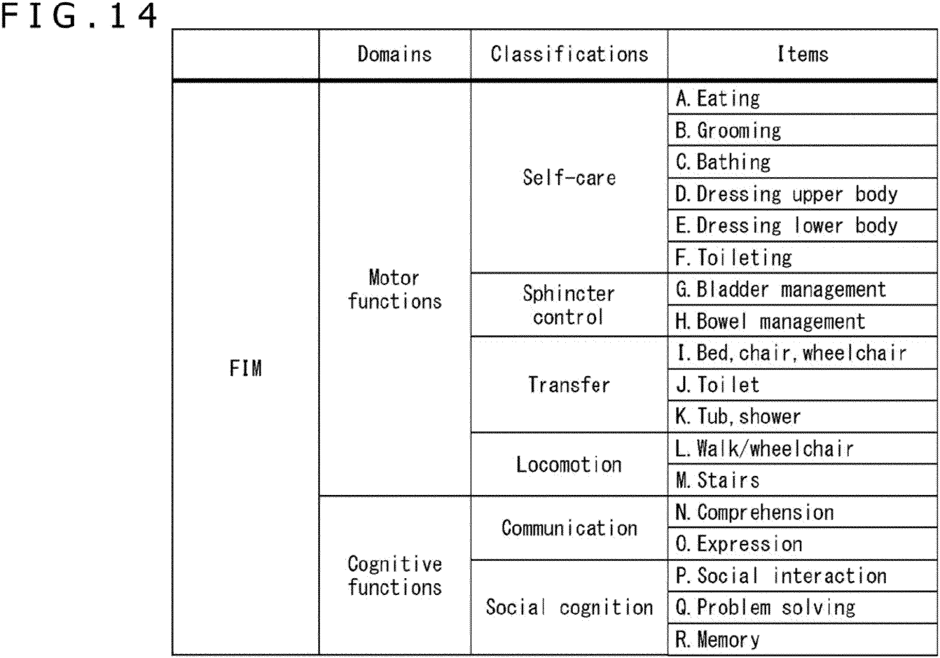

FIG. 14 is a diagram explaining FIM (Functional Independence Measure) as an example of indexes of health benefits.

Even if crude drugs have the same crude drug name, they may have different quantities of beneficial ingredients depending on the production area or the like, in some cases. In addition, even if herbal medicines are produced by the same crude drug blending method such as a blending quantity of crude drugs, they may have different health benefits concerning health such as clinical effects, in some cases.

Moreover, it has been confirmed that a crude drug used for production (formulation) of an herbal medicine, particularly a crude drug (produced from a medicinal plant) cultivated by synecoculture (registered trademark), contains, in addition to beneficial ingredients, other various ingredients (medicinal ingredients) which may offer medicinal effects. It is sometimes unclear whether each ingredient contained in a crude drug is biologically active. Accordingly, whether such an ingredient works (is effective) is unknown unless health benefits such as actual clinical effects are checked.

When an herbal medicine offering a desired health benefit is to be produced, there are following problems: how to evaluate health benefits; and how to predict health benefits of an herbal medicine produced by blending crude drugs having various ingredients.

Indexes of health benefits need to be established to evaluate and predict health benefits. As such indexes, results of bioassay of various organ cells with respect to herbal medicines having different quantities of beneficial ingredients and toxic ingredients, clinical data, and epidemiological data can be used, for example. Moreover, FIM is adoptable as the indexes of health benefits.

FIM is a measure for evaluating how well a person can carry out activities of daily living by him or herself, and is used to evaluate a disability level of a patient and a state change of a patient according to rehabilitation or medical intervention. For example, the details of FIM are described in J.M. Linacre et al. “The Structure and Stability Independence Measure.” Arch phys Med Rahabil Vol75, February 1994.

FIM includes measures of 18 items (Items) of physical, mental, and social functions, i.e., items of “eating,” “grooming,” “bathing,” “dressing upper body,” “dressing lower body,” “toileting,” “bladder management,” “bowel management,” “bed, chair wheelchair,” “toilet,” “tub, shower,” “walk/wheelchair,” “stairs,” “comprehension,” “expression,” “social interaction,” “problem solving,” and “memory.”

FIM is classified into domains of motor functions and domains of cognitive functions.

The domains of the motor functional brain are divided into classifications of self-care, sphincter control, transfer, and locomotion.

The items “eating,” “grooming,” “bathing,” “dressing upper body,” “dressing lower body,” and “toileting” belong to the self-care, while the items “bladder management” and “bowel management” belong to the sphincter control. The items “bed, chair, wheelchair,” “toilet,” and “tub, shower” belong to the transfer, while the items “walk/wheelchair” and “stairs” belong to the locomotion.

The domains of the cognitive functions are divided into classifications of communication and social cognition.

The items “comprehension” and “expression” belong to the communication, while the items “social interaction,” “problem solving,” and “memory” belong to the social cognition.

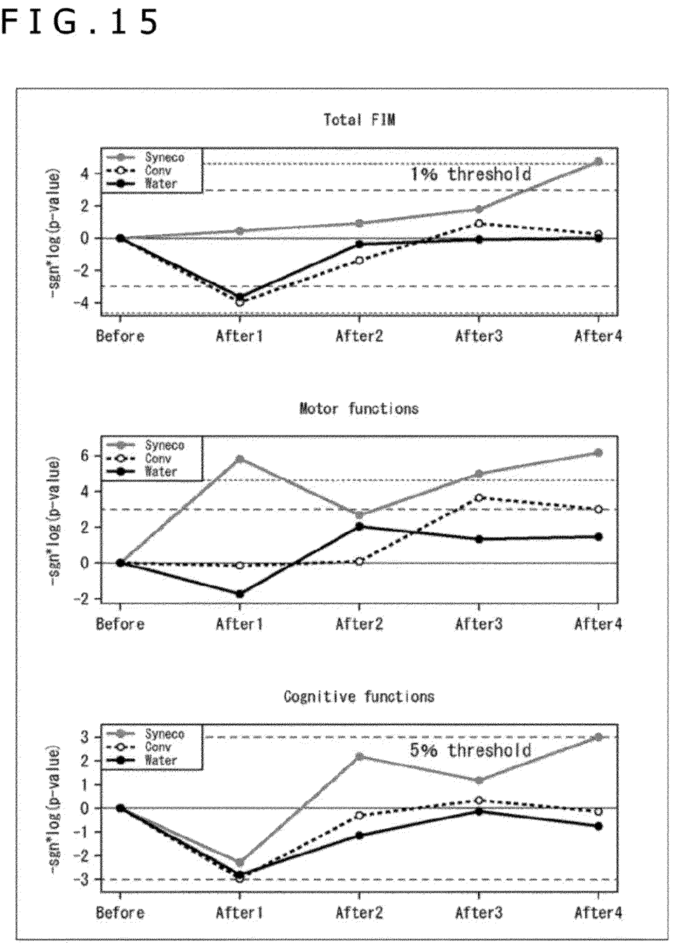

FIG. 15 is a diagram depicting a result of an experiment concerning an FIM improvement as health benefits resulting from ingestion of tea cultivated by synecoculture (registered trademark).

The experiment was performed for 117 persons in total including 45 persons who ingested tea cultivated by synecoculture (registered trademark), 42 persons who ingested tea cultivated by the conventional farming method, and 30 persons who ingested water.

FIG. 15 illustrates transitions of total FIM, FIM in the domains of motor functions, and FIM in the domains of cognitive functions of the persons who had ingested tea cultivated by synecoculture (registered trademark) for four months.

In FIG. 15, in addition to the transition of FIM (Syneco) of the persons who ingested tea cultivated by synecoculture (registered trademark), a transition of FIM (Conv) of the persons who ingested tea cultivated by the conventional farming method, and a transition of FIM (Water) of the persons who ingested water are illustrated. Note that a threshold in FIG. 15 is a significant level used for examination of differences between average values.

As can be seen from FIG. 15, it is confirmed that, in the case of the persons who ingested tea cultivated by synecoculture (registered trademark), both FIM in the domains of motor functions and FIM in the domains of cognitive functions increase, and therefore, total FIM increases. According to the experiment, it has been confirmed that, regarding four out of six young women who ingested tea cultivated by synecoculture (registered trademark), FIM of the 7 items included in the abovementioned 18 items have considerably increased.

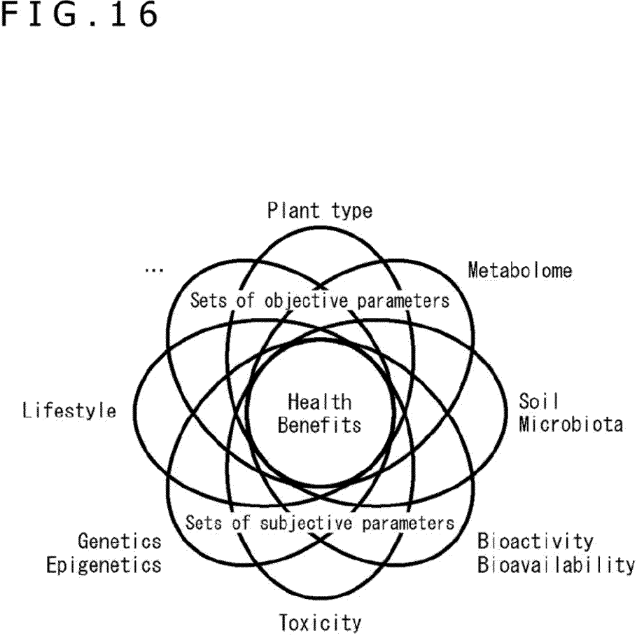

FIG. 16 is a diagram explaining an outline of a health benefit flower model which associates health benefits with herbal medicines.

In the health benefit flower model (second model), health benefits and various parameters associated with (presumed to be associated with) health (hereinafter also referred to as health parameters) are used instead of the crude drugs and the cultivation parameters corresponding to the cultivation conditions, which are used in the crude drug flower model (FIG. 7).

In the health benefit flower model, respective dots in FIG. 16 represent various health benefits (indexes of health benefits), while petals (ellipses) represent factors offering health benefits (hereinafter also referred to as health factors). Each dot within the petal represents a health benefit produced by the health factor represented by the petal.

According to the present technology, big data regarding various health parameters associated with (presumed to be associated with) various health benefits is collected from various persons. Thereafter, the big data regarding the health parameters of the various health benefits is learned by using AI, and a health parameter (type of health parameter and also quantity (value) or the like of health parameter as necessary) significant for offering a health benefit is retrieved as a health factor of the health benefit.

According to the present embodiment, at least information associated with herbal medicines is used as essential health parameters in the health benefit flower model. In such a manner, health benefits and herbal medicines are associated with each other in the health benefit flower model. For example, the information associated with herbal medicines includes types and quantities of herbal medicines, types and quantities of crude drugs contained in herbal medicines, cultivation conditions, types and quantities of ingredients contained in crude drugs, and others.

In the health benefit flower model, the relation between a health benefit and an herbal medicine as a health factor offering the health benefit is expressed in such a form that a dot (a region including the dot) representing the health benefit is included in a petal representing the herbal medicine as the health factor offering the health benefit.

When the health benefit flower model, i.e., the flower model associating a health benefit and an herbal medicine offering the health benefit, is constructed, a petal (an ellipse as the petal) representing a health parameter which may constitute a health factor can be set (added) in an appropriate manner in learning of big data regarding various health parameters including herbal medicines. If the health parameter represented by the petal constitutes a health factor significant for a health benefit, the petal representing the health factor changes in such a manner as to include a dot representing the health benefit affected by the health factor. On the other hand, if the health parameter represented by the petal does not constitute a significant health factor, the corresponding petal disappears.

As the health benefits (indexes of the health benefits) in the health benefit flower model, not only FIM but also various biomarkers, QOL (Quality of Life) (values indicating QOL), and other subjective parameters (Sets of subjective parameters) and objective parameters (Sets of objective parameters) can be used.

The subjective parameters refer to parameters that are measured by a person and that are variable depending on the person who carries out the measurement. Examples of the subjective parameters include text (regardless of whether or not contents of the text are based on an objective phenomenon) created by a person. For example, FIM is measured by such a person as health-care personnel in a clinical site, and is thus the subjective parameter.

The objective parameters refer to parameters measured by a machine, such as output values from a sensor. For example, a heart rate is affected by subjective thinking, but is an objective parameter as long as the heart rate is measured by a heart rate monitor. In addition, for example, a biomarker measured by a machine is an objective parameter.

For example, the details of the subjective parameters and the objective parameters are described in Funabashi, M. “Citizen Science and Topology of Mind: Complexity, Computation and Criticality in Data-Driven Exploration of Open Complex Systems” Entropy 2017, 19, 181. (https://www.mdpi.com/1099-4300/19/4/181).

As the health parameters in the health benefit flower model, in addition to information associated with herbal medicines, various pieces of information associated with (presumed to be associated with) health, such as information associated with lifestyles, living environments, and biomarkers, and information used for diagnostic criteria of diseases (e.g., blood pressure and visceral fat area), can be used.

According to the health benefit flower model, for example, an herbal medicine (information associated with herbal medicine) offering a desired health benefit and other health factors are identifiable.

For example, the flower model of the desired health benefit can be constructed by retrieving a health factor which is represented by a petal including a dot representing the desired health benefit, by using a gradient method.

According to the present technology, a health parameter including at least information associated with an herbal medicine is set as a health parameter which may become a health factor represented by a petal. Thereafter, a health factor (a health parameter corresponding to the health factor) represented by a petal including a dot representing a desired health benefit is retrieved by a gradient method to construct a flower model of the desired health benefit. The flower model is a flower model associating the desired health benefit with a health factor offering the desired health benefit. Moreover, according to the present technology, an herbal medicine, a lifestyle, or the like as a health factor offering the desired health benefit is identified by using such a flower model. When people ingest the herbal medicine identified by the present technology or adopt the lifestyle identified by the present technology, for example, they can gain the desired health benefit with higher reproducibility.

In the flower model depicted in FIG. 16, a range indicated by Health benefits is included in petals representing information such as “Plant type,” “Metabolome,” “Soil Microbiota,” “Bioactivity/Bioavailability,” “Toxicity,” “Genetics/Epigenetics,” and “Lifestyle” as health factors.

Information indicating items “Plant type,” “Metabolome,” “Soil Microbiota,” “Bioactivity/Bioavailability,” and “Toxicity” is information associated with an herbal medicine to be ingested. The item “Plant type” indicates information associated with a plant species as a crude drug (a medicinal plant producing the crude drug) blended in the herbal medicine to be ingested. The item “Metabolome” indicates information associated with an ingredient composition obtained by performing metabolomic analysis for a crude drug (a medicinal plant producing the crude drug) blended in the herbal medicine to be ingested. The item “Soil Microbiota” indicates information associated with soil microbiota of soil where a crude drug (a medicinal plant producing the crude drug) blended in the herbal medicine to be ingested is cultivated. The item “Bioactivity/Bioavailability” indicates information associated with a biological activity and bioavailability of ingredients of the herbal medicine to be ingested (e.g., performance of biological activity when ingredients of the herbal medicine are metabolized). The item “Toxicity” indicates information associated with toxic ingredients of the herbal medicine to be ingested.

The item “Genetics/Epigenetics” indicates information associated with genetic information (genetic information such as genetic disease risks).

The item “Lifestyle” indicates information associated with lifestyles (e.g., smoking habit, exercise habit, and dietary habit).

According to the health benefit flower model, it is possible to not only identify a health factor offering a desired health benefit, such as an herbal medicine and a lifestyle, but also identify health benefits brought to any person by setting petals representing health factors of the person and predict the health benefits assumed to be brought to the person.

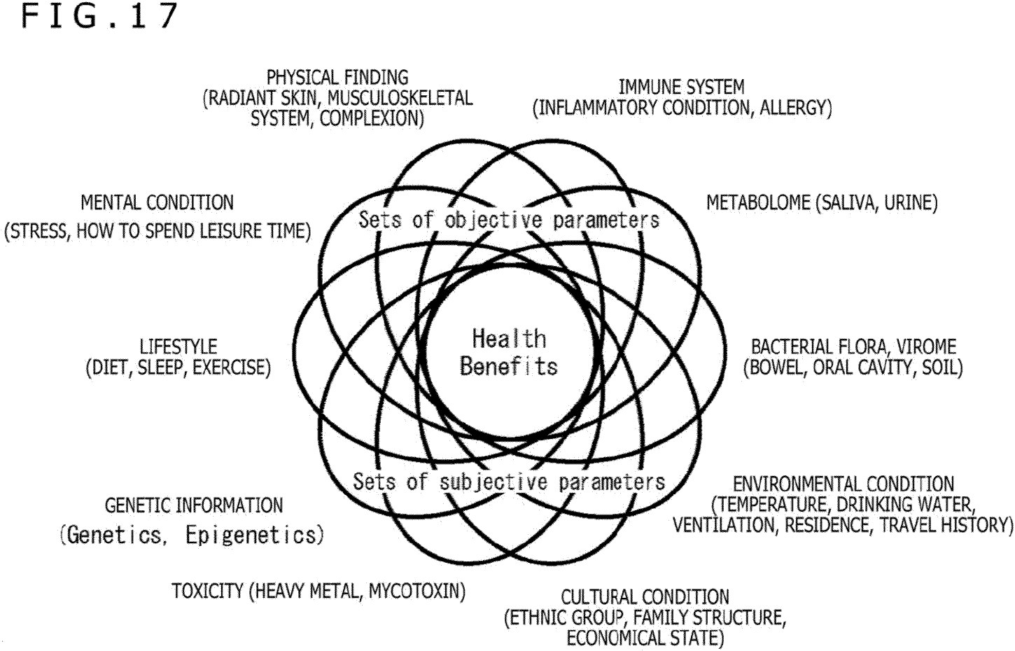

FIG. 17 is a diagram depicting an example of health parameters other than an herbal medicine.

For example, as health parameters other than an herbal medicine in the health benefit flower model, the following pieces of information can be used.

Information associated with immune systems such as inflammatory conditions and allergies
Information associated with metabolomic analysis using saliva, urine, or the like
Information associated with bacterial flora and virome in bowels, oral cavity, soil where ingested food is cultivated, or the like
Information associated with environmental conditions of a living place or the like, such as temperature, a state of drinking water, ventilation, residence, and a travel history
Information associated with cultural conditions such as an ethnic group, a family structure, and an economical state
Information associated with toxicity such as heavy metal and mycotoxin contained in ingested food or the like
Genetic information associated with genetic disease risks or the like (Genetics, Epigenetics)
Information associated with lifestyles such as diet, sleep, and exercise
Information associated with mental conditions such as stress and how to spend leisure time
Information associated with physical findings such as a skin condition, musculoskeletal systems, and a complexion

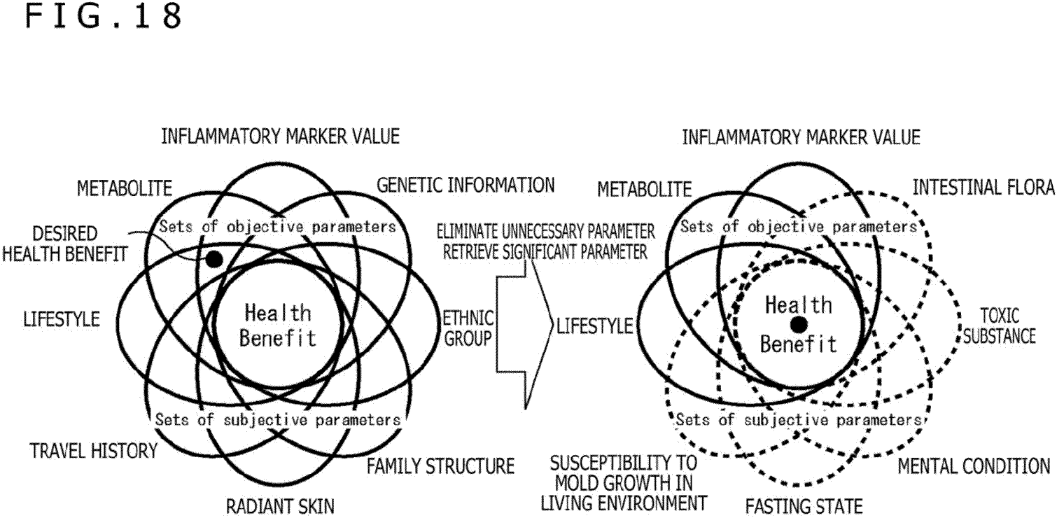

FIG. 18 is a diagram depicting an example of construction of a flower model of a desired health benefit.

A flower model on the left of FIG. 18 represents a flower model which has petals (ellipses) each indicating a current health factor (a health parameter corresponding to the current health factor) of a target person who desires to increase a desired health benefit.

According to the left flower model, information associated with a value of an inflammatory marker, genetic information, an ethnic group, a family structure, a skin condition, a travel history, a lifestyle, and metabolite represents (is presumed to be) current health factors of the target person.

In the left flower model, a dot representing the desired health benefit deviates from a range indicated by Health Benefit where all of the current health factors of the target person overlap with each other.

Upon the construction of the flower model of the desired health benefit, in order to provide the desired health benefit, unnecessary health parameters are eliminated, and significant health parameters are retrieved as a health factor of the desired health benefit. Thus, the flower model that includes petals (ellipses) each including the dot representing the desired health benefit is constructed.

A flower model on the right of FIG. 18 is a flower model constructed by retrieval of the health factor of the desired health benefit.

In the right flower model, genetic information, information associated with an ethnic group, information associated with a family structure, information associated with a skin condition, and information associated with a travel history, which are cultivation conditions included in the left flower model, are eliminated (disappear) since they are cultivation parameters unnecessary to obtain the desired health benefit.

Moreover, in the right flower model, in addition to the information associated with an inflammatory marker value, the information associated with a lifestyle, and the information associated with metabolite, which are the health factors included in the left flower model, information associated with intestinal flora, information associated with a toxic substance, information associated with a mental condition, information associated with a fasting state, and information associated with susceptibility to mold growth in a living environment, which are not included in the left flower model (and are indicated by dotted ellipses in the figure), are retrieved as health parameters significant for the desired health benefit.

Note that, although not illustrated in FIG. 18, information associated with an herbal medicine is set as a health parameter, and a health factor including the information associated with the herbal medicine is retrieved in the present technology.

Moreover, it is known that an inflammatory marker value, a result of metabolomic analysis (metabolite), and information associated with a lifestyle are significant health parameters. It is also known that intestinal flora, a toxic substance, a mental condition, a fasting state, and susceptibility to mold growth in a living environment considerably contribute to development of a disease.

### <Production of Herbal Medicine>

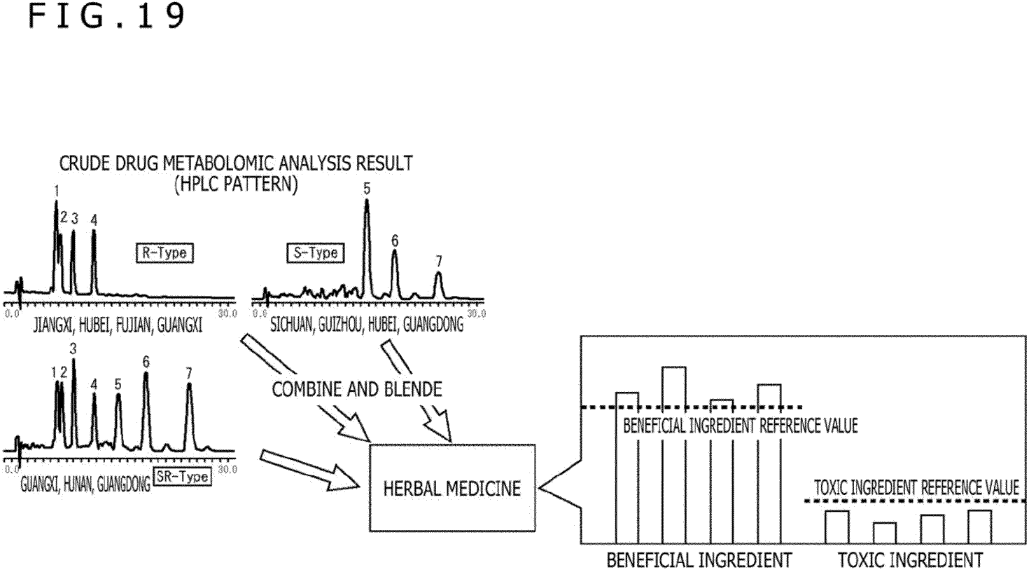

FIG. 19 is a diagram explaining blending of crude drugs to produce an herbal medicine.

For example, as explained with reference to FIGS. 12 and 13, ingredients of a crude drug such as Uncaria Hook differ depending on the production area. In a case where a certain quantity of crude drugs is sold as a lot, ingredients of the crude drugs differ between the production area on a lot basis.

In order to produce an herbal medicine, crude drugs which are produced in different areas on a lot basis and which have multiple different ingredients depending on their production areas are combined and blended. Through such blending, the herbal medicine is produced such that beneficial ingredients thereof are maintained equal to or above a reference value of the beneficial ingredients and toxic ingredients thereof are maintained equal to or below a reference value of the toxic ingredients. Here, the beneficial ingredients and the toxic ingredients may include identical substances. In such a case, these ingredients act on a living body as either beneficial ingredients or toxic ingredients depending on the concentration of the ingredients.

Moreover, a flower model of a desired health benefit is constructed by using a result of bioassay performed on a person ingesting an herbal medicine or the like, a clinical effect, or other information, and an herbal medicine (information associated with the herbal medicine) corresponding to a health factor offering the desired health benefit is identified by using the flower model.

In identification of the herbal medicine corresponding to the health factor offering the desired health benefit, the relation between the herbal medicine (a crude drug blended to produce the herbal medicine) and the desired health benefit is obtained and fed back to a production process of the herbal medicine.

Then, in production of the herbal medicine, a crude drug blending quantity is calculated by using linear programming or non-linear programming such that beneficial ingredients are equal to or above a reference value and toxic ingredients are equal to or below a reference value, and such that the objective function, which is obtained from the relation between the herbal medicine and the desired health benefit and represents a change of the desired health benefit with respect to the blending quantity of the crude drugs contained in the herbal medicine, is maximized.

The identification of the herbal medicine corresponding to the health factor offering the desired health benefit, the feedback of the relation between the herbal medicine (crude drug) and the desired health benefit, and the calculation of the crude drug blending quantity which maximizes the objective function obtained from the relation between the herbal medicine and the desired health benefit and representing the desired health benefit are repeatedly carried out. In such a manner, accuracy of the identification of the herbal medicine corresponding to the factor offering the desired health benefit and accuracy of the calculation of the crude drug blending quantity which maximizes the objective function representing a change of the desired health benefit are improved.

In a case where accurate calculation of such a crude drug blending quantity that maximizes the objective function representing a change of the desired health benefit is achieved, and where a blending quantity newly calculated is considered (substantially) similar to the blending quantity previously calculated, a crude drug portfolio in which the crude drug blending quantity newly calculated is registered can be constructed.

An herbal medicine is produced with the crude drugs blended according to the crude drug portfolio, and thus, quality management can be conducted to ensure the quality of the produced herbal medicine at a level equal to (or above) a fixed level.

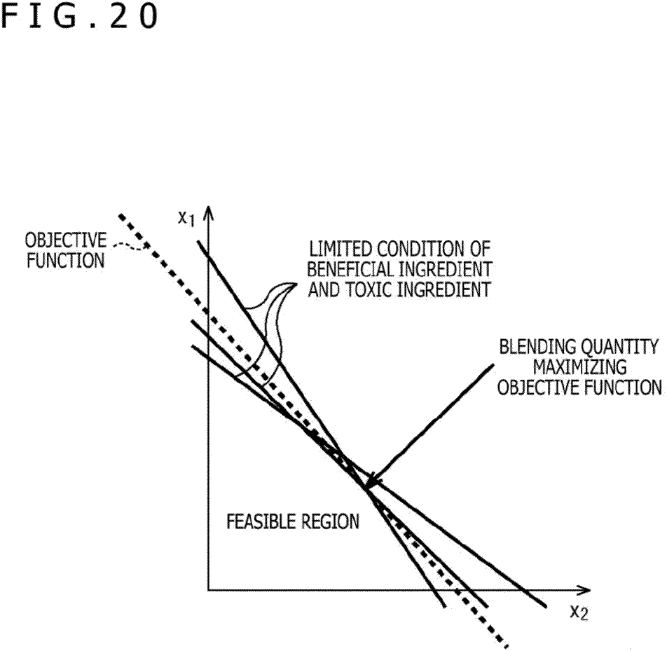

FIG. 20 is a diagram depicting an example in which a crude drug blending quantity is calculated by using linear programming such that beneficial ingredients are equal to or above a reference value and toxic ingredients are equal to or below a reference value and such that an objective function representing a change of a desired health benefit is maximized.

For example, the details of linear programming are described in https://ja.wikipedia.org/wiki/%E7%B7%9A%E5%9E%8B%E8%A8%88%E 7%94%BB%E6%B3%95.

FIG. 20 is a graph that is represented in a two-dimensional space (plane) and includes two axes representing two crude drug blending quantities x1 and x2. The crude drug blending quantities x1 and x2 are blending quantities of crude drugs that are blended to produce an herbal medicine corresponding to a health factor offering a desired health benefit. The graph also includes lines (indicated by solid lines in FIG. 20) that indicate a limited condition where beneficial ingredients are equal to or above a reference value and a limited condition where toxic ingredients are equal to or below a reference value, and a line (indicated by a dotted line in FIG. 20) that indicates an objective function representing a change of the desired health benefit with respect to the blending quantity of the crude drugs contained in the herbal medicine.

In linear programming, such crude drug blending quantities that maximize the objective function representing a change of the desired health benefit, such as the two crude drug blending quantities x1 and x2 in FIG. 20, are calculated in a feasible region where the limited condition where the beneficial ingredients are equal to or above the reference value and the limited condition where the toxic ingredients are equal to or below the reference value are both met.

In a case where any two or more ingredients included in the beneficial ingredients and the toxic ingredients of the crude drug used for production of the herbal medicine do not interact with each other in a blending process of the crude drug or in pharmacokinetics after ingestion of the herbal medicine, the crude drug blending quantity having the beneficial ingredients equal to or above the reference value and the toxic ingredients equal to or below the reference value and maximizing the objective function representing a change of the desired health benefit can be calculated by using linear programming.

On the other hand, in a case where any two or more ingredients included in the beneficial ingredients and the toxic ingredients of the crude drug used for production of the herbal medicine interact with each other, the objective function, the limited conditions, or both the objective function and the limited conditions become non-linear. In such a case, the crude drug blending quantity which has the beneficial ingredients equal to or above the reference value and the toxic ingredients equal to or below the reference value and maximizes the objective function representing a change of the desired health benefit needs to be calculated by using non-linear programming in consideration of non-linearity.

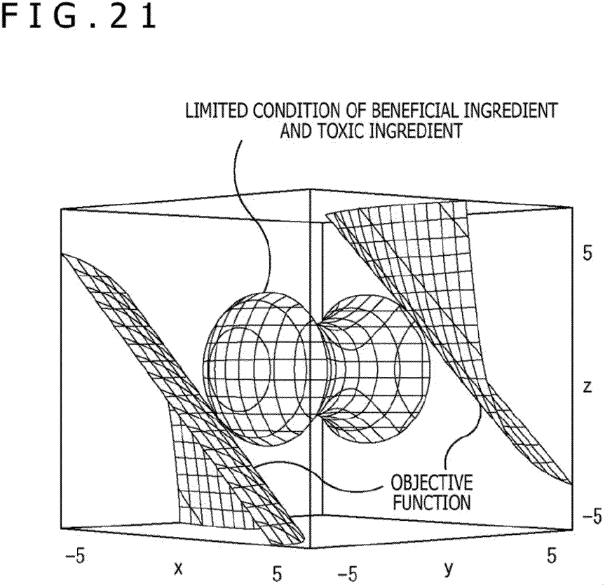

FIG. 21 is a diagram depicting an example in which a crude drug blending quantity is calculated by using non-linear programming such that beneficial ingredients are equal to or above a reference value and toxic ingredients are equal to or below a reference value and such that the objective function representing a change of a desired health benefit is maximized.

For example, the details of non-linear programming are described in https://ja.wikipedia.org/wiki/%E9%9D%9E%E7%B7%9A%E5%BD%A2%E 8%A8%88%E7%94%BB%E6%B3%95.

FIG. 21 is a graph that is represented in a three-dimensional space and includes three axes representing three crude drug blending quantities x, y, and z. The crude drug blending quantities x, y, and z are blending quantities of crude drugs that are blended to produce an herbal medicine corresponding to a health factor offering a desired health benefit. The graph also includes a three-dimensional diagram that indicates a limited condition where beneficial ingredients are equal to or above a reference value and toxic ingredients are equal to or below a reference value, and planar diagrams that indicate the objective function representing a change of the desired health benefit.

Non-linear interactions between ingredients of the crude drug or the like, which are caused by blending of the crude drugs at the time of production of the herbal medicine or as a result of digestion absorption of the herbal medicine, are modeled on the basis of big data, and the obtained model can be used to calculate the three-dimensional diagram representing the limited condition and the planar diagram representing the objective function.

In non-linear programming, a point of contact between the three-dimensional diagram representing the limited condition where beneficial ingredients are equal to or above the reference value and toxic ingredients are equal to or below the reference value, and the planar diagram representing the objective function is calculated as a crude drug blending quantity which maximizes the objective function. In FIG. 21, the three crude drug blending quantities x, y, and z which maximize the objective function are calculated.

For example, the point of contact between the three-dimensional diagram representing the limited condition and the planar diagram representing the objective function can be calculating by using AI or the like to perform fitting of the three-dimensional diagram representing the limited condition and the planar diagram representing the objective function.

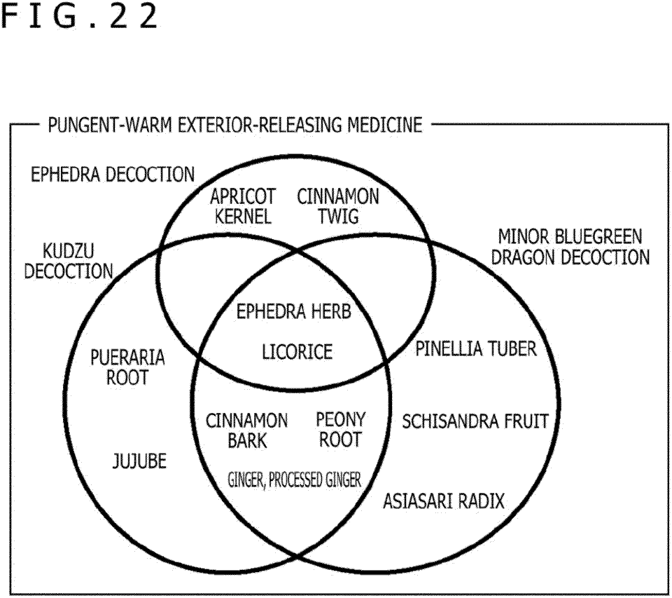

FIG. 22 is a diagram depicting crude drugs blended to produce herbal medicines that are classified into a pungent-warm exterior-releasing medicine.

FIG. 22 illustrates crude drugs that are blended according to a classic prescription using kudzu decoction, ephedra decoction, and minor bluegreen dragon decoction, which are classified into the pungent-warm exterior-releasing medicine, in such a manner that these crude drugs are included in corresponding ellipses. The classic prescription refers to a prescription described in Chinese classics.

Incidentally, as described with reference to FIGS. 12 and 13, crude drugs that have the same crude drug name may have different ingredient compositions depending on the production area, in some cases. Moreover, even though crude drugs are produced in the same area, a previous crude drug and a current crude drug may have different ingredient compositions due to a change of cultivation conditions.

In a case where crude drugs having the same crude drug name but having different ingredient compositions as described above are blended according to types and quantities of a classic prescription, a health benefit (efficacy) that a person who ingests an herbal medicine produced by such blending of the crude drugs will gain is presumed to deviate from a health benefit originally expected to be offered from the herbal medicine, depending on ingredient compositions of the crude drugs.

On the other hand, a new formulation for blending crude drugs offering a desired health benefit (information associated with a crude drug or the like, such as a production area or a cultivation condition of the crude drug, the type of crude drug, a processing method, and a blending quantity) can be generated by identifying, with the use of the health benefit flower model, information associated with a crude drug or the like (including a food and a drink that are not currently defined as a crude drug but are expected to produce a medicinal effect) corresponding to a health factor offering a desired health benefit, and calculating, with the use of non-linear programming or the like, such a blending quantity of crude drugs or the like that meets the reference values of beneficial ingredients and toxic ingredients and that maximizes the objective function representing a change of the desired health benefit. Such a new formulation is also referred to as a generative prescription.

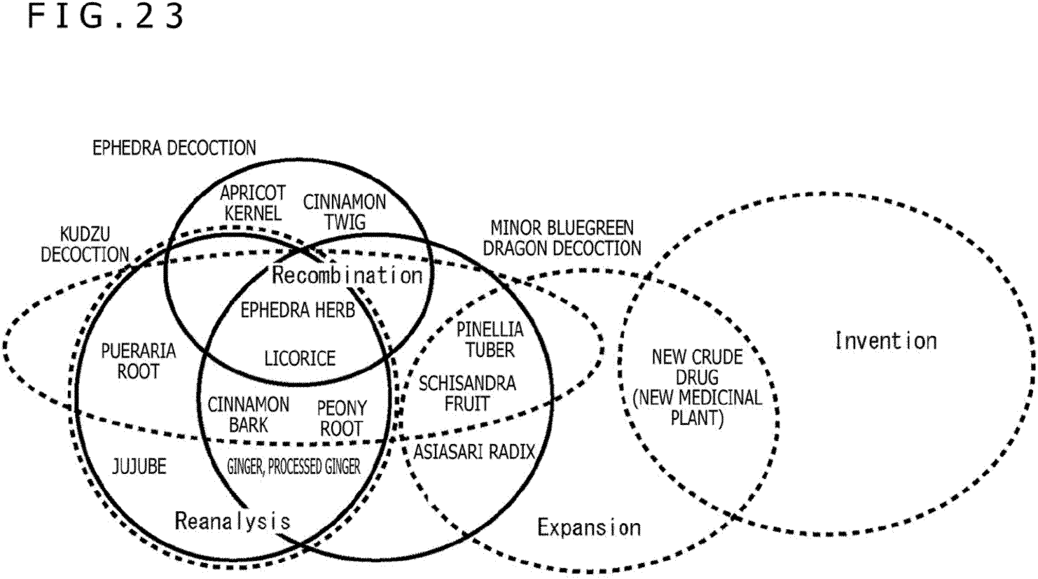

FIG. 23 is a diagram depicting an example of a generative prescription.

In FIG. 23, ellipses indicated by solid lines represent crude drugs blended according to a classic prescription, while ellipses indicated by dotted lines represent crude drug blended according to a generative prescription.

For example, as a generative prescription, it is possible to establish a crude drug blending method capable of offering a health benefit which should be offered from Kudzu Decoction produced according to a classic prescription, i.e., information associated with crude drugs which are to be blended according to a classic prescription to produce Kudzu Decoction and which have different ingredient compositions or the like depending on their production areas, cultivation conditions, or the like, and associated with what production area and cultivation condition are adopted to cultivate such crude drugs and how much quantity of crude drugs is blended so as to offer a greater health benefit than that of the blending method described in a classic prescription, for example (Reanalysis).

Moreover, as a generative prescription, it is possible to establish a blending method for producing a new and more effective herbal medicine as a pungent-warm exterior-releasing medicine by blending crude drugs (e.g., licorice) common to kudzu decoction, ephedra decoction, and minor bluegreen dragon decoction, which are classified into the pungent-warm exterior-releasing medicine, with other crude drugs, for example (Recombination).

Further, as a generative prescription, it is possible to establish a blending method for producing a new herbal medicine by blending a new crude drug with an existing crude drug (Expansion).

In addition, as a generative prescription, it is possible to establish a blending method for producing a new herbal medicine by blending only new crude drugs (Invention).

For example, the new crude drug herein may be discovered by identifying, with the use of the health benefit flower model, information associated with a food and a drink corresponding to a health factor which offers a desired health benefit.

For example, as depicted in FIG. 15, in a case where tea is cultivated by synecoculture (registered trademark) (hereinafter the tea is also referred to as synecoculture (registered trademark) tea), FIM as a health benefit of the tea is improved. Accordingly, such synecoculture (registered trademark) tea can be identified as a health factor which offers a specific health benefit, by using a health benefit flower model. In a case where synecoculture (registered trademark) tea is identified as a health factor offering a specific health benefit, the synecoculture (registered trademark) tea is considered to have a medicinal effect contributing to the specific health benefit, and can thus be recognized as a new crude drug different from tea cultivated by the conventional farming method.

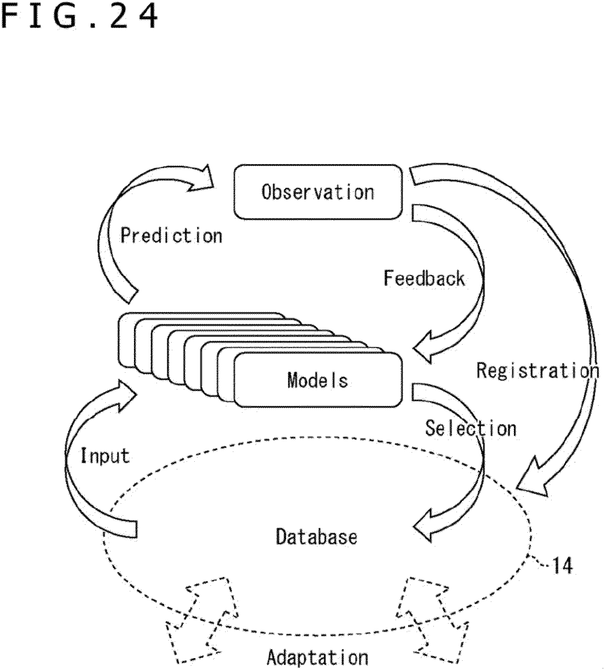

FIG. 24 is a diagram explaining a framework of dynamic real-time management of a super-diversity management system.

The super-diversity management system is implemented in the server 13. The super-diversity management system is a system which dynamically manages data (information) indicating biodiversity and other various types of diversity, on a real time basis, in order to achieve quality management (quality control) of herbal medicines and establish a generative prescription.

Examples of data to be subjected to the dynamic real-time management (dynamic real-time management of data associated with biodiversity and other various types of diversity) includes data regarding multi-omics, data regarding biodiversity in farm fields using synecoculture (registered trademark), data regarding bioassay and clinical trials (clinical effects) for persons who ingest an herbal medicine, tea cultivated by synecoculture (registered trademark), or other foods and drinks and for persons who do not ingest them, data regarding a classic prescription, data regarding cultivation conditions of crude drugs, data regarding processing conditions of crude drugs, data regarding health benefits, data regarding lifestyles, and data regarding a result of metabolomic analysis of ingredients of a crude drug.

FIG. 24 depicts the framework of the dynamic real-time management performed by the super-diversity management system.

Various types of observations are conducted by the sensor device 11, the terminal 12, and the like (Observation). Observation values obtained as a result of the observations (e.g., sensor data resulting from the sensing by the sensor device 11, text input by the user operating the terminal 12, and captured images) are registered in the database 14 as necessary (Registration). In addition, data regarding multi-omics, data regarding biodiversity, data regarding bioassay or clinical trials, data regarding a classic prescription, and other various types of data are registered in the database 14 as necessary.

Multiple models such as various types of mathematical models including a machine learning model and a statistical mathematical model are implemented in the super-diversity management system (Models). The multiple models are learned by AI using data registered in the database 14.

The super-diversity management system predicts various observation values, i.e., acquires prediction values (Prediction), by inputting data registered in the database 14 to respective learned models (Input).

The super-diversity management system receives feedback of actual observation values (Feedback), and compares the actual observation values with the corresponding prediction values.

Thereafter, the super-diversity management system determines significance of models and data registered in the models and the database, according to a result of comparison between the actual observation values and the prediction values, and selects any of models and data registered in the models and the database, according to a result of the determination.

For example, in a case where a difference between the actual observation values and the prediction values meets a condition such as a threshold set beforehand, the models and the data registered in the models and the database are determined to be significant. On the other hand, in a case where a difference between the actual observation values and the prediction values does not meet the condition such as a threshold set beforehand, the models and the data registered in the models or the database are not determined to be significant.

The super-diversity management system deletes (discards) the models not significant from the multiple models, and holds (extracts and uses) the significant models.

Moreover, the super-diversity management system deletes the data not significant from the data registered in the database 14, and holds the significant data.

Adaptation of the database 14, i.e., collection of significant data, is achieved (Adaptation) by registration of observation values in the database 14 and selection of data registered in the database 14 (Selection).

By using significant data collected to the database 14, the server 13 performs construction of a crude drug flower model, construction of a health benefit flower model, calculation of crude drug blending quantity using non-linear programming, or the like, for example.

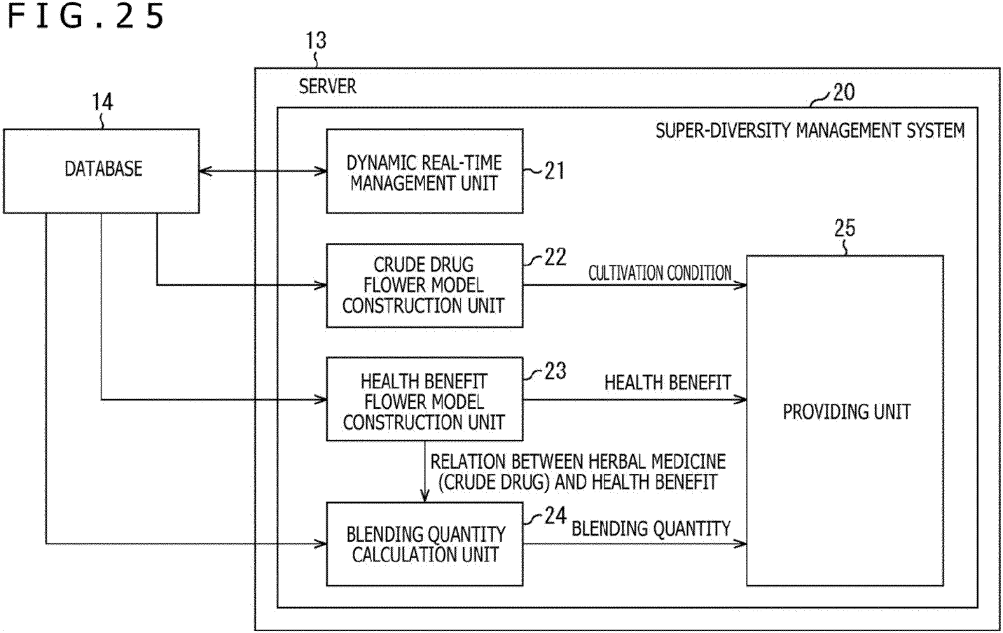

FIG. 25 is a block diagram depicting a functional configuration example of the server 13.

A super-diversity management system 20 is implemented in the server 13.

The super-diversity management system 20 includes a dynamic real-time management unit 21, a crude drug flower model construction unit 22, a health benefit flower model construction unit 23, a blending quantity calculation unit 24, and a providing unit 25.

The dynamic real-time management unit 21 handles the dynamic real-time management described with reference to FIG. 24, to collect significant data to the database 14.

The crude drug flower model construction unit 22 functions as a first identification unit that identifies, with the use of a crude drug flower model, a cultivation condition for cultivating a specific crude drug (a medicinal plant producing the crude drug) by synecoculture (registered trademark).

In the crude drug flower model, the crude drug flower model construction unit 22 sets cultivation parameters that include at least a parameter associated with synecoculture (registered trademark) and included in significant data registered in the database 14, such as a yield of a crude drug (a medicinal plant producing the crude drug) cultivated by synecoculture (registered trademark), a quantity of solar radiation in a farm field where the crude drug is cultivated, diversity of soil microorganisms, types of mixed plants, a height of ridges, and a soil quality.

Moreover, in the crude drug flower model, the crude drug flower model construction unit 22 uses the significant data registered in the database 14 to retrieve, by a gradient method, a cultivation condition represented by a petal including a dot indicating a specific crude drug designated by the user operating the terminal 12 or the like, i.e., a cultivation parameter (a value of the parameter) corresponding to the cultivation condition for cultivating the specific crude drug, for example, from the set cultivation parameters. Thus, a flower model of the specific crude drug is constructed.

Thereafter, the crude drug flower model construction unit 22 uses the flower model of the specific crude drug to identify the cultivation parameter represented by the petal of the flower model, as a cultivation condition for cultivating the specific crude drug with high reproducibility, and supplies the identified cultivation condition to the providing unit 25. The cultivation condition supplied from the crude drug flower model construction unit 22 to the providing unit 25 includes a cultivation condition for cultivating the specific crude drug by synecoculture (registered trademark).

The health benefit flower model construction unit 23 functions as a second identification unit that identifies, with the use of a health benefit flower model, a health factor such as an herbal medicine and a lifestyle offering a specific health benefit.

In the health benefit flower model, the health benefit flower model construction unit 23 sets health parameters that include at least parameters associated with an herbal medicine and a lifestyle as necessary and included in significant data registered in the database 14, such as herbal medicines including an herbal medicine whose blending quantity is calculated by the blending quantity calculation unit 24, effects of bioassay and clinical trials on persons ingesting the herbal medicine, FIM, and a lifestyle.

Moreover, in the health benefit flower model, the health benefit flower model construction unit 23 uses the significant data registered in the database 14 to retrieve, by a gradient method, a health factor represented by a petal including a dot indicating a specific health benefit designated by the user operating the terminal 12 or the like, i.e., a health parameter corresponding to the health factor for offering the specific health benefit, for example, from the set health parameters. Thus, a flower model of the specific health benefit is constructed.

Thereafter, the health benefit flower model construction unit 23 uses the flower model of the specific health benefit to identify the health parameter represented by the petal of the flower model, as a health factor for offering the specific health benefit with high reproducibility, and supplies the identified health factor to the providing unit 25. The health factor supplied from the health benefit flower model construction unit 23 to the providing unit 25 includes an herbal medicine (information associated with the herbal medicine) and also a lifestyle (information associated with the lifestyle) as necessary.

Moreover, when identifying an herbal medicine or the like corresponding to a health factor offering a specific health benefit, the health benefit flower model construction unit 23 feeds back (supplies), to the blending quantity calculation unit 24, the relation between an herbal medicine (a crude drug blended to produce the herbal medicine) and a health benefit, which is obtained at the time of the construction of the flower model of the specific health benefit.

The blending quantity calculation unit 24 uses significant data registered in the database 14, such as a result of metabolomic analysis of each crude drug cultivated in a production area or a farm field and the reference values of beneficial ingredients and toxic ingredients, to calculate a blending quantity of crude drugs to be used to produce an herbal medicine, the quantity having beneficial ingredients equal to or above the reference value and toxic ingredients equal to or below the reference value, and supplies the calculated blending quantity to the providing unit 25.

Moreover, the blending quantity calculation unit 24 uses significant data registered in the database 14 to calculate, by linear programming or non-linear programming, a crude drug blending quantity that has beneficial ingredients equal to or above a reference value and toxic ingredients equal to or below a reference value and that maximizes the objective function representing a change of a specific health benefit with respect to the crude drug blending quantity, the crude drug blending quantity being based on a classic prescription or a generative prescription, and supplies the calculated crude drug blending quantity to the providing unit 25. The objective function representing a change of the desired health benefit is obtained from the relation between the herbal medicine and the health benefit which is fed back from the health benefit flower model construction unit 23.

The providing unit 25 provides the cultivation condition received from the crude drug flower model construction unit 22, the cultivation condition being used to cultivate a specific crude drug (a medicinal plant producing the crude drug) and including a cultivation condition associated with synecoculture (registered trademark), the health factor received from the health benefit flower model construction unit 23, the health factor offering a specific health benefit and including an herbal medicine or a lifestyle, and the crude drug blending quantity received from the blending quantity calculation unit 24.

For example, the providing unit 25 displays the cultivation condition, the health factor, and the crude drug blending quantity according to an operation performed by the supporter using the server 13.

Moreover, for example, the providing unit 25 transmits the cultivation condition, the health factor, and the crude drug blending quantity to the terminal 12 according to an operation performed by the user using the terminal 12, and causes the terminal 12 to display them.

Accordingly, the super-diversity management system 20 can provide a cultivation condition for cultivating a specific crude drug desired by the user with high reproducibility, a health factor such as an herbal medicine and a lifestyle for offering a specific health benefit desired by the user with high reproducibility, and a crude drug blending quantity for producing an herbal medicine offering a specific health benefit with high reproducibility.

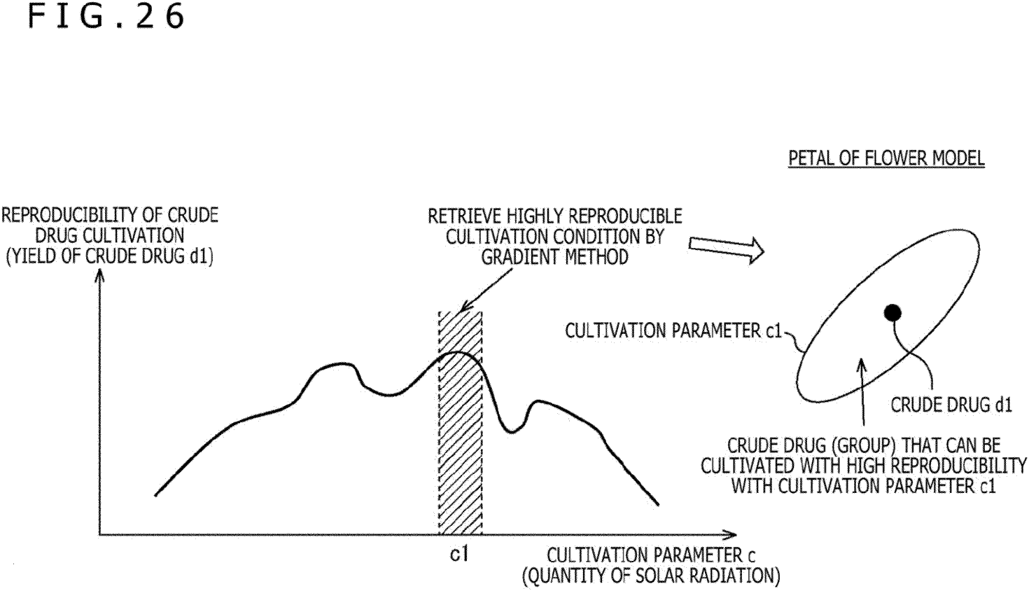

FIG. 26 is a diagram explaining an outline of construction of a crude drug flower model which is performed by the crude drug flower model construction unit 22.

The crude drug flower model construction unit 22 uses a cultivation parameter c, e.g., a quantity of solar radiation, registered as significant data in the database 14 and a yield of a crude drug d1 as reproducibility of cultivation of the crude drug d1, to retrieve, by a gradient method, a value (a range of the value) c1 of the cultivation parameter c, e.g., the quantity of solar radiation, at which the reproducibility of cultivation of the crude drug d1 is maximized. Thus, the crude drug flower model construction unit 22 constructs a flower model of the crude drug d1.

In the flower model of the crude drug d1, an ellipse (indicated by a solid line in FIG. 26) including a dot representing a crude drug that can be cultivated with high reproducibility at the value c1 of the cultivation parameter c, that is, the quantity of solar radiation herein, at which the reproducibility of cultivation of the crude drug d1 is maximized, is illustrated as one petal of the flower model.

The crude drug d1 (the dot representing the crude drug d1) is included in the petal expressed by the ellipse including the dot representing the crude drug that can be cultivated with high reproducibility at the value c1 of the cultivation parameter c, i.e., the quantity of solar radiation.

Construction of the health benefit flower model by the health benefit flower model construction unit 23 is performed similarly to the construction of the crude drug flower model by the crude drug flower model construction unit 22.

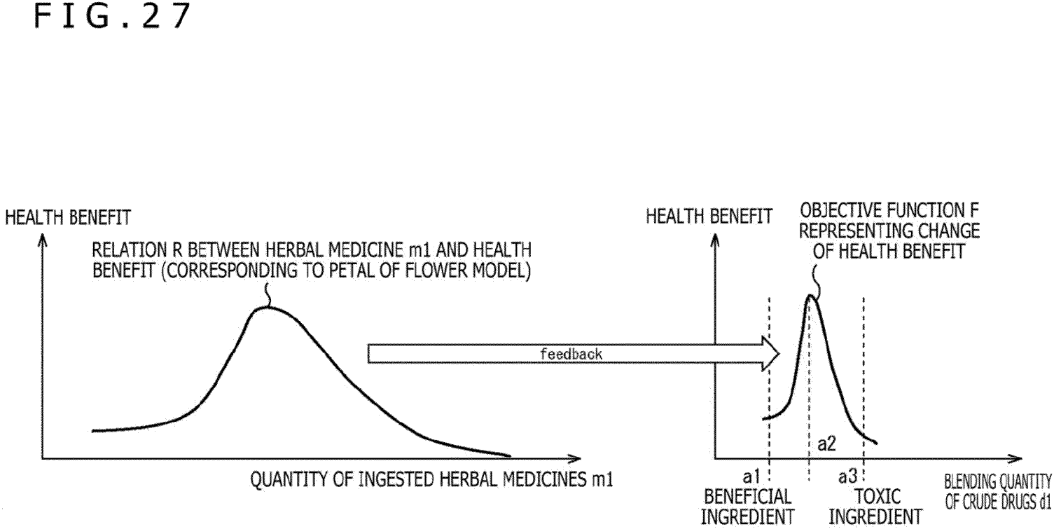

FIG. 27 is a diagram explaining an outline of calculation of a crude drug blending quantity which is performed by the blending quantity calculation unit 24.

For example, the health benefit flower model construction unit 23 retrieves, by a gradient method, a quantity of ingested herbal medicines m1 in which FIM or the like as a specific health benefit is maximized. Here, the quantity of ingested herbal medicines m1 is a health parameter. Thus, a flower model of the specific health benefit is constructed. The health benefit flower model construction unit 23 obtains a relation R between the herbal medicine m1 (the quantity of the ingested herbal medicine m1) and the specific health benefit, in the construction of the flower model of the specific health benefit. The relation R is fed back to the blending quantity calculation unit 24.

As a quantity of the crude drugs d1 blended to produce the herbal medicine m1, the blending quantity calculation unit 24 calculates, by using non-linear programming or the like, a blending quantity a2 of the crude drugs d1 in which an objective function F that is obtained from the relation R between the herbal medicine m1 and the specific health benefit, which is fed back from the health benefit flower model construction unit 23, and that represents a change of the specific health benefit is maximized in a range of a blending quantity a1 having beneficial ingredients of the reference value to a blending quantity a3 having toxic ingredients of the reference value.

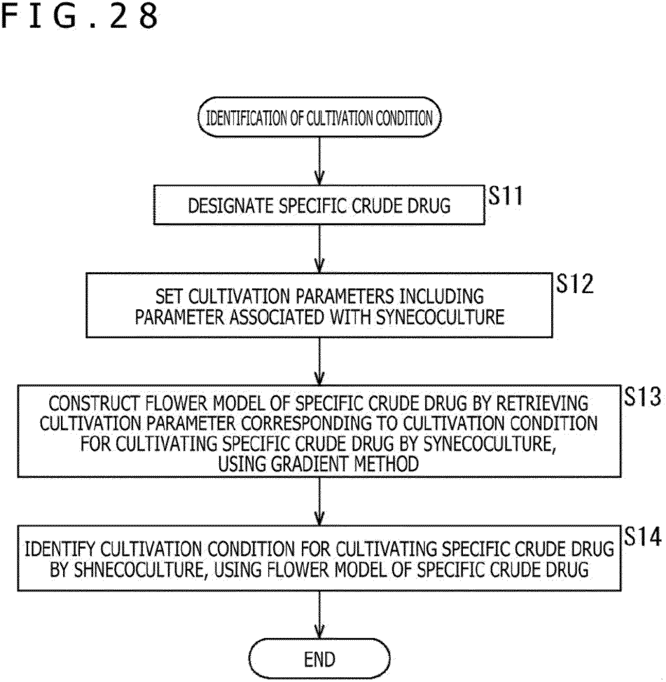

FIG. 28 is a flowchart explaining an example of a process of identifying a cultivation condition by the crude drug flower model construction unit 22.

In step S11, the crude drug flower model construction unit 22 designates a crude drug desired by a user, as a specific crude drug, according to an operation performed by the user using the terminal 12, for example. The process then proceeds to step S12.

In step S12, in a crude drug flower model, the crude drug flower model construction unit 22 sets cultivation parameters that include at least a parameter associated with synecoculture (registered trademark) and included in significant data registered in the database 14. The process then proceeds to step S13.

In step S13, in the crude drug flower model, the crude drug flower model construction unit 22 uses the significant data registered in the database 14 to retrieve, by a gradient method, a cultivation parameter corresponding to a cultivation condition for cultivating the specific crude drug by synecoculture (registered trademark), from the set cultivation parameters. Thus, the crude drug flower model construction unit 22 constructs a flower model of the specific crude drug. The process then proceeds to step S14.

In step S14, the crude drug flower model construction unit 22 uses the flower model of the specific crude drug to identify the cultivation condition for cultivating the specific crude drug by synecoculture (registered trademark), and supplies the identified cultivation condition. Thereafter, the process ends.

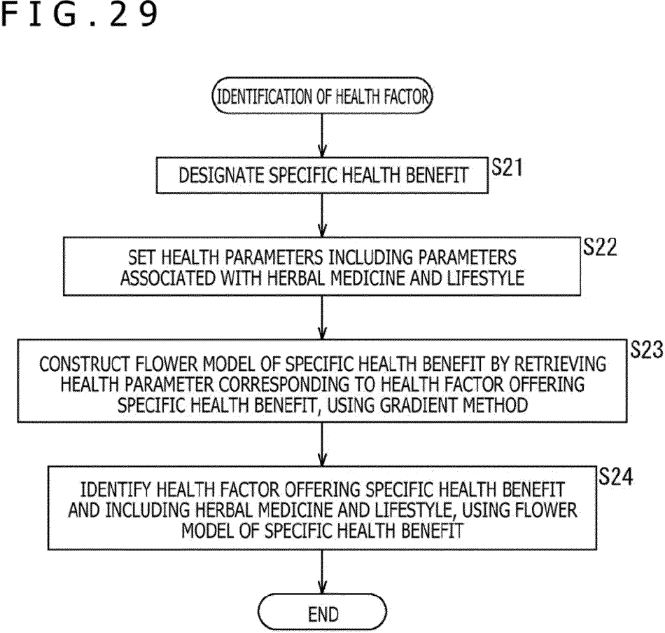

FIG. 29 is a flowchart explaining an example of a process of identifying a health factor by the health benefit flower model construction unit 23.

In step S21, the health benefit flower model construction unit 23 designates a health benefit desired by a user, as a specific health benefit, according to an operation performed by the user using the terminal 12, for example. The process then proceeds to step S22.

In step S22, the health benefit flower model construction unit 23 sets health parameters that include at least parameters associated with an herbal medicine and a lifestyle and included in significant data registered in the database 14. The process then proceeds to step S23.

In step S23, in the health benefit flower model, the health benefit flower model construction unit 23 uses the significant data registered in the database 14 to retrieve, by a gradient method, a health parameter corresponding to a health factor offering the specific health benefit, from the set health parameters. Thus, the health benefit flower model construction unit 23 constructs a flower model of the specific health benefit. The process then proceeds to step S24.

In step S24, the health benefit flower model construction unit 23 uses the flower model of the specific health benefit to identify the health parameter offering the specific health benefit and including the herbal medicine and the lifestyle, and supplies the identified health factor to the providing unit 25. Thereafter, the process ends.

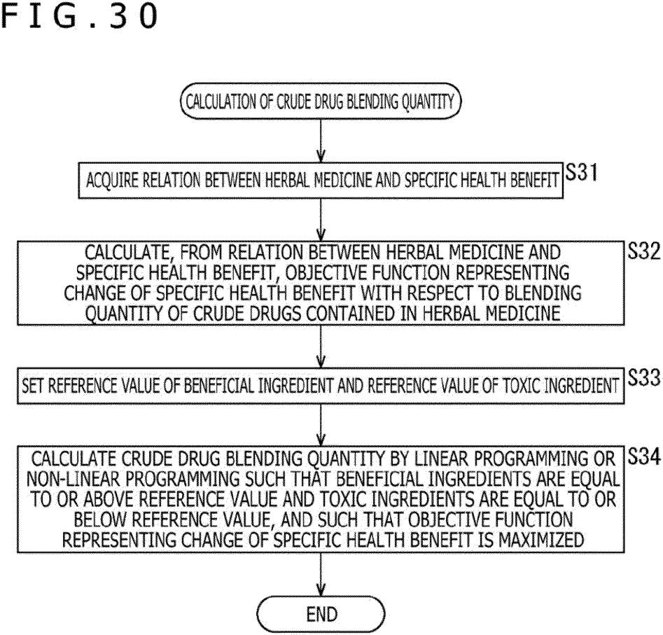

FIG. 30 is a flowchart explaining an example of a process of calculating a crude drug blending quantity by the blending quantity calculation unit 24.

In step S31, the blending quantity calculation unit 24 acquires (receives) the relation between an herbal medicine and a specific health benefit from the health benefit flower model construction unit 23. The process then proceeds to step S32.

In step S32, the blending quantity calculation unit 24 calculates, from the relation between the herbal medicine and the specific health benefit, an objective function representing a change of the specific health benefit with respect to a blending quantity of crude drugs contained in the herbal medicine. The process then proceeds to step S33.

In step S33, the blending quantity calculation unit 24 uses the significant data registered in the database 14 to set a reference value of beneficial ingredients and a reference value of toxic ingredients. The process then proceeds to step S34.

In step S34, the blending quantity calculation unit 24 calculates a blending quantity of the crude drugs by linear programming or non-linear programming such that beneficial ingredients are equal to or above the reference value and toxic ingredients are equal to or below the reference value and such that the objective function representing the change of the specific health benefit is maximized, and supplies the calculated quantity to the providing unit 25. Thereafter, the process ends.

### <Details of Computer to Which Present Technology Is Applied>

A series of processes performed by the terminal 12 or the server 13 as described above may be executed by either hardware or software. In a case where the series of processes are executed by software, a program included in the software is installed in a computer functioning as the terminal 12 or the server 13.

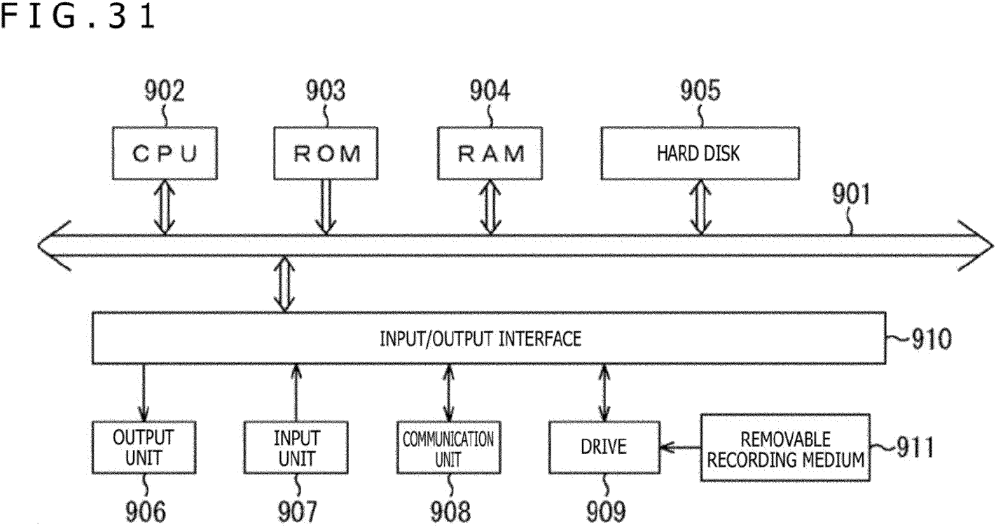

FIG. 31 is a block diagram depicting a configuration example of a computer according to one embodiment where a program for executing the series of processes described above is installed, i.e., a hardware configuration example of the terminal 12 and the server 13.

The program can be recorded beforehand in a hard disk 905 or a ROM 903 as a recording medium built in the computer.

Alternatively, the program can be stored (recorded) in a removable recording medium 911 driven by a drive 909. The removable recording medium 911 storing the program can be provided as what is called package software. Examples of the removable recording medium 911 herein include a flexible disc, a CD-ROM (Compact Disc Read Only Memory), an MO (Magneto Optical) disc, a DVD (Digital Versatile Disc), a magnetic disc, and a semiconductor memory.

Note that the program can be installed into the computer from the removable recording medium 911 described above, or otherwise be downloaded to the computer via a communication network or a broadcasting network and installed into the built-in hard disk 905. In other words, for example, the program can wirelessly be transferred to the computer from a download site via an artificial satellite for digital satellite broadcasting, or can be transferred by wire to the computer via a network such as a LAN (Local Area Network) and the Internet.

The computer includes a built-in CPU (Central Processing Unit) 902. An input/output interface 910 is connected to the CPU 902 via a bus 901.

When a command is input to the CPU 902 via the input/output interface 910 from a user using an input unit 907, for example, the CPU 902 executes the program stored in the ROM (Read Only Memory) 903 according to the command. Alternatively, the CPU 902 loads the program stored in the hard disk 905 to a RAM (Random Access Memory) 904, and executes the program.

In such a manner, the CPU 902 performs the processes according to the flowcharts described above or the configurations of the block diagrams described above. Thereafter, the CPU 902 causes an output unit 906 to output a result of the processes, causes a communication unit 908 to transmit the result, or causes the hard disk 905 to record the result, for example, via the input/output interface or the like, as necessary.

Note that the input unit 907 includes a keyboard, a mouse, a microphone, and the like. Further, the output unit 906 includes an LCD (Liquid Crystal Display), a speaker, and the like.

Note herein that the processes performed by the computer according to the program in the present description are not necessarily required to be executed in time series in the orders described in the flowcharts. In other words, the processes performed by the computer according to the program include processes executed in parallel or individually (e.g., parallel processing or processing by object).

Moreover, the program may be processed by one computer (processor) or may be processed in a distributed manner by using multiple computers. In addition, the program may be transferred to and executed by a remote computer.

Further, the system in the present description refers to a set of multiple constituent elements (e.g., devices, modules (parts)) regardless of whether or not all constituent elements are disposed in an identical housing. Accordingly, a system including multiple devices disposed in separate housings and connected to one another via a network, and a system including one device having multiple modules disposed in one housing are both systems.

Note that the embodiments of the present technology are not limited to the embodiments described above, and may be modified in various manners without departing from the scope of the subject matters of the present technology.

For example, the present technology may have a configuration of cloud computing where one function is shared and processed by multiple devices in cooperation with each other via a network.

Moreover, the respective steps described in the above flowcharts can be executed by one device or can be shared and executed by multiple devices.

Further, in a case where multiple processes are included in one step, the multiple processes included in the one step can be executed by one device, or can be shared and executed by multiple devices.

In addition, the advantageous effects described in the present description are presented only by way of example without a limitation on these. Other advantageous effects may be offered.

Note that the present technology can adopt the following configurations.

<1> An information processing device including:

a first identification unit that identifies a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.

<2> The information processing device according to <1>, in which the first identification unit constructs the first model by retrieving, according to a gradient method, a cultivation parameter corresponding to the cultivation condition for cultivating the specific crude drug, from cultivation parameters that are associated with cultivation of crude drugs and that include a parameter associated with the diversity-promoting cultivation method.

<3> The information processing device according to <2>, in which the parameter associated with the diversity-promoting cultivation method includes one or more pieces of information regarding a quantity of solar radiation, diversity of soil microorganisms, types of mixed vegetation, a height of a ridge, an amount of water in soil, and drainage of soil.

<4> The information processing device according to any one of <1> to <3>, further including:

a second identification unit that identifies a health factor offering a specific health benefit and including an herbal medicine, with use of a second model that associates a health benefit with a health factor offering the health benefit and including an herbal medicine.

<5> The information processing device according to <4>, in which the second identification unit constructs the second model by retrieving, according to a gradient method, a health parameter corresponding to the health factor offering the specific health benefit, from health parameters that are associated with health and that include a parameter associated with an herbal medicine.

<6> The information processing device according to <4> or <5>, in which the second identification unit identifies health factors offering the specific health benefit and including an herbal medicine and a lifestyle.

<7> The information processing device according to <6>, in which the second identification unit constructs the second model by retrieving, according to a gradient method, a health parameter corresponding to the health factor offering the specific health benefit, from the health parameters that include parameters associated with an herbal medicine and a lifestyle.

<8> The information processing device according to any one of <4> to <7>, further including:

a blending quantity calculation unit that calculates a blending quantity of crude drugs that are to be blended to produce an herbal medicine such that beneficial ingredients are equal to or above a reference value of the beneficial ingredients and toxic ingredients are equal to or below a reference value of the toxic ingredients.

<9> The information processing device according to <8>, in which the blending quantity calculation unit calculates the blending quantity of the crude drugs by linear programming or non-linear programming such that the beneficial ingredients are equal to or above the reference value of the beneficial ingredients and the toxic ingredients are equal to or below the reference value of the toxic ingredients, and such that an objective function representing a change of the specific health benefit with respect to the blending quantity of the crude drugs is maximized.

<10> The information processing device according to <9>, in which the objective function is obtained on the basis of a relation between herbal medicines and health benefits, the relation being acquired by construction of the second model.

<11> An information processing method including:

identifying a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.

<12> A program for causing a computer to function as:

a first identification unit that identifies a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.

[Reference Signs List]

| | |
|---|---|
| 11: | Sensor device |
| 12: | Terminal |
| 13: | Server |
| 14: | Database |
| 20: | Super-diversity management system |
| 21: | Dynamic real-time management unit |
| 22: | Crude drug flower model construction unit |
| 23: | Health benefit flower model construction unit |
| 24: | Blending quantity calculation unit |
| 25: | Providing unit |
| 901: | Bus |
| 902: | CPU |
| 903: | ROM |
| 904: | RAM |
| 905: | Hard disk |
| 906: | Output unit |
| 907: | Input unit |
| 908: | Communication unit |
| 909: | Drive |
| 910: | Input/output interface |
| 911: | Removable recording medium |

1. An information processing device comprising:
a first identification unit that identifies a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.

2. The information processing device according to claim 1, wherein the first identification unit constructs the first model by retrieving, according to a gradient method, a cultivation parameter corresponding to the cultivation condition for cultivating the specific crude drug, from cultivation parameters that are associated with cultivation of crude drugs and that include a parameter associated with the diversity-promoting cultivation method.

3. The information processing device according to claim 2, wherein the parameter associated with the diversity-promoting cultivation method includes one or more pieces of information regarding a quantity of solar radiation, diversity of soil microorganisms, types of mixed vegetation, a height of a ridge, an amount of water in soil, and drainage of soil.

4. The information processing device according to claim 1, further comprising:
a second identification unit that identifies a health factor offering a specific health benefit and including an herbal medicine, with use of a second model that associates a health benefit with a health factor offering the health benefit and including an herbal medicine.

5. The information processing device according to claim 4, wherein the second identification unit constructs the second model by retrieving, according to a gradient method, a health parameter corresponding to the health factor offering the specific health benefit, from health parameters that are associated with health and that include a parameter associated with an herbal medicine.

6. The information processing device according to claim 4, wherein the second identification unit identifies health factors offering the specific health benefit and including an herbal medicine and a lifestyle.

7. The information processing device according to claim 6, wherein the second identification unit constructs the second model by retrieving, according to a gradient method, a health parameter corresponding to the health factor offering the specific health benefit, from the health parameters that include parameters associated with an herbal medicine and a lifestyle.

8. The information processing device according to claim 4, further comprising:
a blending quantity calculation unit that calculates a blending quantity of crude drugs that are to be blended to produce an herbal medicine such that beneficial ingredients are equal to or above a reference value of the beneficial ingredients and toxic ingredients are equal to or below a reference value of the toxic ingredients.

9. The information processing device according to claim 8, wherein the blending quantity calculation unit calculates the blending quantity of the crude drugs by linear programming or non-linear programming such that the beneficial ingredients are equal to or above the reference value of the beneficial ingredients and the toxic ingredients are equal to or below the reference value of the toxic ingredients, and such that an objective function representing a change of the specific health benefit with respect to the blending quantity of the crude drugs is maximized.

10. The information processing device according to claim 9, wherein the objective function is obtained on a basis of a relation between herbal medicines and health benefits, the relation being acquired by construction of the second model.

11. An information processing method comprising:
identifying a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.

12. A program for causing a computer to function as:
a first identification unit that identifies a cultivation condition for cultivating a specific crude drug, with use of a first model that associates a crude drug with a cultivation condition for cultivating the crude drug by a diversity-promoting cultivation method for promoting biodiversity and controlling an ecosystem to produce vegetation.
